SEQUENCE LISTING (55 sequences)

SEQ ID NO: 1 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDFKGLTPLHLAAAHGHLEIVEVLLKAGADVNAKDVYGWTPLHIAAASGHLEIVEVLLKAGADVNAKDWLGITPLHLAASHGHLEIVEVLLKAGADVNAQDKSGKTPADLAARAGHQDIAEVLQKAA

SEQ ID NO: 2 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDFKGLTPLHLAAAHGHLEIVEVLLKAGADVNAKDVYGWTPLHWAAASGHLEIVEVLLKAGADVNAKDWLGITPLHLAASHGHLEIVEVLLKAGADVNAQDKSGKTPADLAARAGHQDIAEVLQKAA

SEQ ID NO: 3 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDFKGLTPLHLAAAHGHLEIVEVLLKAGADVNAKDVYGWTPLHWAAAKGHLEIVEVLLKAGADVNAKDWLGITPLHLAASHGHLEIVEVLLKAGADVNAQDKSGKTPADLAARAGHQDIAEVLQKAA

SEQ ID NO: 4 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDFKGLTPLHLAAEHGHLEIVEVLLKAGADVNAKDVYGWTPLHWAAAKGHLEIVEVLLKAGADVNAKDWLGITPLHLAASHGHLEIVEVLLKAGADVNAQDKSGKTPADLAARAGHQDIAEVLQKAA

SEQ ID NO: 5 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDHYGWTPLHIAAQIGHLEIVEVLLKAGADVNAKDWIGYTPLHLAASHGHLEIVEVLLKAGADVNAKDVSGKTPLHVAAAHGHLEIVEVLLKAGADVNAQDKSGKTPADLAARAGHQDIAEVLQKAA

SEQ ID NO: 6 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDQYGWTPLHLAAYSGHLEIVEVLLKAGADVNAKDWVGWTPLHLAASHGHLEIVEVLLKAGADVNAKDEAGRTPLHIAAKQGHLEIVEVLLKAGADVNAQDKSGKTPADLAARAGHQDIAEVLQKAA

SEQ ID NO: 7 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDHYGWTPLHLAAAEGHLEIVEVLLKAGADVNAKDWIGYTPLHIAASHGHLEIVEVLLKAGADVNAKDSSGKTPLHIAAQHGHLEIVEVLLKAGADVNAQDKSGKTPADLAARAGHQDIAEVLQKAA

SEQ ID NO: 8 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDHYGWTPLHIAAQRGHLEIVEVLLKAGADVNAKDWLGWTPLHVAASHGHLEIVEVLLKAGADVNAKDLSGRTPLHIAARQGHLEIVEVLLKAGADVNAQDKSGKTPADLAARAGHQDIAEVLQKAA

SEQ ID NO: 9 - protein, 159 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDHYGWTPLHLAASEGHLEIVEVLLKAGADVNAKDWIGWTPLHLAASFGHLEIVEVLLKAGADVNAKDVSGKTPLHIAARQGHLEIVEVLLKAGADVNAQDKSGKTPADLAARAGHQDIAEVLQLQKAA

SEQ ID NO: 10 - protein, 157 aa, Artificial Sequence
  source: /note=“Description of Artificial Sequence Synthetic polypeptide”
  source: /note=“Designed ankyrin repeat domain”
DLGKKLLQAARAGQLDEVRELLKAGADVNAKDHYGWTPLHLAASEGHLEIVEVLLKAGADVNAKDWIGWTPLHLAASFGHLEIVEVLLKAGADVNAKDVSGKTPLHIAARQGHLEIVEVLLKAGADVNAQDKSGKTPADLAARAGHQDIAEVLQKAA

SEQ ID NO: 11 - protein, 124 aa, Artificial Sequence
  source: /note=“Description of Artificial Sequence Synthetic polypeptide”
  source: /note=“CD3-specific binding domain”
DLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAA

SEQ ID NO: 12 - protein, 124 aa, Artificial Sequence
  source: /note=“Description of Artificial Sequence Synthetic polypeptide”
  source: /note=“CD3-specific binding domain”
DLGQKLLEAAWAGQDDEVRELLKAGADVNAKDSQGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 13 - protein, 124 aa, Artificial Sequence
  source: /note=“Description of Artificial Sequence Synthetic polypeptide”
  source: /note=“CD3-specific binding domain”
DLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 14 - protein, 124 aa, Artificial Sequence
  source: /note=“Description of Artificial Sequence Synthetic polypeptide”
  source: /note=“CD3-specific binding domain”
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 15 - protein, 124 aa, Artificial Sequence
  source: /note=“Description of Artificial Sequence Synthetic polypeptide”
  source: /note=“CD3-specific binding domain”
DLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 16 - DNA, 471 bp, Artificial Sequence
  source: /note=“Description of Artificial Sequence Synthetic polynucleotide”
  source: /note=“Nucleic acid encoding a designed ankyrin repeat domain”
gacttaggaaagaaattgctgcaagccgcacgcgccggtcaacttgatgaggtgcgcgaattattgaaggcaggtgcagacgtgaacgctaaagactttaagggacttactcctttacacttagcggccgcacatggtcatttggaaattgtggaggtcctgttgaaggctggcgccgacgtgaacgccaaagatgtttacggttggaccccattacacattgctgccgcctcgggacatctggaaattgttgaggttctgcttaaagctggcgcagacgttaatgccaaggactggttggggattacgcccttacacctggccgcgtcacatggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 17 - DNA, 471 bp, Artificial Sequence
  source: /note=“Description of Artificial Sequence Synthetic polynucleotide”
  source: /note=“Nucleic acid encoding a designed ankyrin repeat domain”
gacttaggaaagaaattgctgcaagccgcacgcgccggtcaacttgatgaggtgcgcgaattattgaaggcaggtgcagacgtgaacgctaaagactttaagggacttactcctttacacttagcggccgagcatggtcatttggaaattgtggaggtcctgttgaaggctggcgccgacgtgaacgccaaagatgtttacggttggaccccattacactgggctgccgccaagggacatctggaaattgttgaggttctgcttaaagctggcgcagacgttaatgccaaggactggttggggattacgcccttacacctggccgcgtcacatggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 18 - DNA, 477 bp, Artificial Sequence
  source: /note=“Description of Artificial Sequence Synthetic polynucleotide”
  source: /note=“Nucleic acid encoding a designed ankyrin repeat domain”
gacttggggaaaaaactgcttcaggctgcacgcgctggtcagttagatgaggtgcgtgagttgttaaaagctggagcggacgtaaatgctaaagatcattacggatggacacccctgcatcttgcagcgtcagaaggtcaccttgaaatcgtcgaggtccttttgaaagcaggggcagatgtcaacgccaaggactggatcggttggacccctcttcatttagctgcttcgttcggtcatctggagattgtagaagttttattaaaagccggtgcggatgtgaatgcaaaagatgtctcagggaaaaccccgttacacatcgccgctcgtcaagggcatttagagatcgtcgaggtactgttgaaagcgggcgcagatgtcaatgcacaggacaagtccggcaaaactccagcggatttagctgcgcgcgcaggacaccaagacattgcggaagtcttacaactgcagaaggcagcg

SEQ ID NO: 19 - DNA, 471 bp, Artificial Sequence
  source: /note=“Description of Artificial Sequence Synthetic polynucleotide”
  source: /note=“Nucleic acid encoding a designed ankyrin repeat domain”
gacttggggaaaaaactgcttcaggctgcacgcgctggtcagttagatgaggtgcgtgagttgttaaaagctggagcggacgtaaatgctaaagatcattacggatggacacccctgcatcttgcagcgtcagaaggtcaccttgaaatcgtcgaggtccttttgaaagcaggggcagatgtcaacgccaaggactggatcggttggacccctcttcatttagctgcttcgttcggtcatctggagattgtagaagttttattaaaagccggtgcggatgtgaatgcaaaagatgtctcagggaaaaccccgttacacatcgccgctcgtcaagggcatttagagatcgtcgaggtactgttgaaagcgggcgcagatgtcaatgcacaggacaagtccggcaaaactccagcggatttagctgcgcgcgcaggacaccaagacattgcggaagtcctgcagaaggcagcg

SEQ ID NO: 20 - protein, 12 aa, Artificial Sequence
  source: /note=“Description of Artificial Sequence Synthetic peptide”
  source: /note=“His-tag”
MRGSHHHHHHGS

SEQ ID NO: 21 - protein, 30 aa, Artificial Sequence
  source: /note=“Description of Artificial Sequence Synthetic polypeptide”
  source: /note=“N-terminal capping module”
DLGKKLLQAARAGQLDEVRELLKAGADVNA

SEQ ID NO: 22 - protein, 30 aa, Artificial Sequence
  source: /note=“Description of Artificial Sequence Synthetic polypeptide”
  source: /note=“N-terminal capping module”
DLGKKLLQAARAGQLDEVRILLKAGADVNA

SEQ ID NO: 23 - protein, 30 aa, Artificial Sequence
  source: /note=“Description of Artificial Sequence Synthetic polypeptide”
  source: /note=“N-terminal capping module”
DLGKKLLQAARAGQLDEVRILLAAGADVNA

SEQ ID NO: 24 - protein, 30 aa, Artificial Sequence
  source: /note=“Description of Artificial Sequence Synthetic polypeptide”
  source: /note=“N-terminal capping module”
  MOD_RES (4)..(4): Any amino acid
  MOD_RES (11)..(12): Any amino acid
DLGXKLLQAAXXGQLDEVRILLAAGADVNA

SEQ ID NO: 25 - protein, 33 aa, Artificial Sequence
  source: /note=“Description of Artificial Sequence Synthetic polypeptide”
  source: /note=“Ankyrin repeat module”
  MOD_RES (1)..(1): Any amino acid
  MOD_RES (3)..(4): Any amino acid
  MOD_RES (6)..(6): Any amino acid
  MOD_RES (14)..(15): Any amino acid
  MOD_RES (27)..(27): Any amino acid
XDXXGXTPLHLAAXXGHLEIVEVLLKXGADVNA

SEQ ID NO: 26 - protein, 33 aa, Artificial Sequence
  source: /note=“Description of Artificial Sequence Synthetic polypeptide”
  source: /note=“Ankyrin repeat module”
  MOD_RES (3)..(4): Any amino acid
  MOD_RES (6)..(6): Any amino acid
  MOD_RES (14)..(15): Any amino acid
KDXXGXTPLHLAAXXGHLEIVEVLLKAGADVNA

SEQ ID NO: 27 - protein, 33 aa, Artificial Sequence
  source: /note=“Description of Artificial Sequence Synthetic polypeptide”
  source: /note=“Ankyrin repeat module”
  MOD_RES (3)..(4): Any amino acid
  MOD_RES (6)..(6): Any amino acid
  MOD_RES (11)..(11): Any amino acid
  MOD_RES (14)..(15): Any amino acid
KDXXGXTPLHXAAXXGHLEIVEVLLKAGADVNA

SEQ ID NO: 28 - protein, 28 aa, Artificial Sequence
  source: /note=“Description of Artificial Sequence Synthetic peptide”
  source: /note=“C-terminal capping module”
QDKSGKTPADLAARAGHQDIAEVLQKAA

SEQ ID NO: 29 - protein, 28 aa, Artificial Sequence
  source: /note=“Description of Artificial Sequence Synthetic peptide”
  source: /note=“C-terminal capping module”
  MOD_RES (1)..(1): Any amino acid
  MOD_RES (3)..(4): Any amino acid
  MOD_RES (6)..(6): Any amino acid
  MOD_RES (11)..(11): Any amino acid
  MOD_RES (15)..(15): Any amino acid
XDXXGXTPADXAARXGHQDIAEVLQKAA

SEQ ID NO: 30 - protein, 33 aa, Artificial Sequence
  source: /note=“Description of Artificial Sequence Synthetic polypeptide”
  source: /note=“Ankyrin repeat module”
KDFKGLTPLHLAAAHGHLEIVEVLLKAGADVNA

SEQ ID NO: 31 - protein, 33 aa, Artificial Sequence
  source: /note=“Description of Artificial Sequence Synthetic polypeptide”
  source: /note=“Ankyrin repeat module”
KDVYGWTPLHIAAASGHLEIVEVLLKAGADVNA

SEQ ID NO: 32 - protein, 33 aa, Artificial Sequence
  source: /note=“Description of Artificial Sequence Synthetic polypeptide”
  source: /note=“Ankyrin repeat module”
KDWLGITPLHLAASHGHLEIVEVLLKAGADVNA

SEQ ID NO: 33 - protein, 33 aa, Artificial Sequence
  source: /note=“Description of Artificial Sequence Synthetic polypeptide”
  source: /note=“Ankyrin repeat module”
KDVYGWTPLHWAAASGHLEIVEVLLKAGADVNA

SEQ ID NO: 34 - protein, 33 aa, Artificial Sequence
  source: /note=“Description of Artificial Sequence Synthetic polypeptide”
  source: /note=“Ankyrin repeat module”
KDVYGWTPLHWAAAKGHLEIVEVLLKAGADVNA

SEQ ID NO: 35 - protein, 33 aa, Artificial Sequence
  source: /note=“Description of Artificial Sequence Synthetic polypeptide”
  source: /note=“Ankyrin repeat module”
KDFKGLTPLHLAAEHGHLEIVEVLLKAGADVNA

SEQ ID NO: 36 - protein, 33 aa, Artificial Sequence; identical to SEQ ID NO: 34
  source: /note=“Description of Artificial Sequence Synthetic polypeptide”
  source: /note=“Ankyrin repeat module”

SEQ ID NO: 37 - protein, 33 aa, Artificial Sequence
  source: /note=“Description of Artificial Sequence Synthetic polypeptide”
  source: /note=“Ankyrin repeat module”
KDHYGWTPLHIAAQIGHLEIVEVLLKAGADVNA

SEQ ID NO: 38 - protein, 33 aa, Artificial Sequence
  source: /note=“Description of Artificial Sequence Synthetic polypeptide”
  source: /note=“Ankyrin repeat module”
KDWIGYTPLHLAASHGHLEIVEVLLKAGADVNA

SEQ ID NO: 39 - protein, 33 aa, Artificial Sequence
  source: /note=“Description of Artificial Sequence Synthetic polypeptide”
  source: /note=“Ankyrin repeat module”
KDVSGKTPLHVAAAHGHLEIVEVLLKAGADVNA

SEQ ID NO: 40 - protein, 33 aa, Artificial Sequence
  source: /note=“Description of Artificial Sequence Synthetic polypeptide”
  source: /note=“Ankyrin repeat module”
KDQYGWTPLHLAAYSGHLEIVEVLLKAGADVNA

SEQ ID NO: 41 - protein, 33 aa, Artificial Sequence
  source: /note=“Description of Artificial Sequence Synthetic polypeptide”
  source: /note=“Ankyrin repeat module”
KDWVGWTPLHLAASHGHLEIVEVLLKAGADVNA

SEQ ID NO: 42 - protein, 33 aa, Artificial Sequence
  source: /note=“Description of Artificial Sequence Synthetic polypeptide”
  source: /note=“Ankyrin repeat module”
KDEAGRTPLHIAAKQGHLEIVEVLLKAGADVNA

SEQ ID NO: 43 - protein, 33 aa, Artificial Sequence
  source: /note=“Description of Artificial Sequence Synthetic polypeptide”
  source: /note=“Ankyrin repeat module”
KDHYGWTPLHLAAAEGHLEIVEVLLKAGADVNA

SEQ ID NO: 44 - protein, 33 aa, Artificial Sequence
  source: /note=“Description of Artificial Sequence Synthetic polypeptide”
  source: /note=“Ankyrin repeat module”
KDWIGYTPLHIAASHGHLEIVEVLLKAGADVNA

SEQ ID NO: 45 - protein, 33 aa, Artificial Sequence
  source: /note=“Description of Artificial Sequence Synthetic polypeptide”
  source: /note=“Ankyrin repeat module”
KDSSGKTPLHIAAQHGHLEIVEVLLKAGADVNA

SEQ ID NO: 46 - protein, 33 aa, Artificial Sequence
  source: /note=“Description of Artificial Sequence Synthetic polypeptide”
  source: /note=“Ankyrin repeat module”
KDHYGWTPLHIAAQRGHLEIVEVLLKAGADVNA

SEQ ID NO: 47 - protein, 33 aa, Artificial Sequence
  source: /note=“Description of Artificial Sequence Synthetic polypeptide”
  source: /note=“Ankyrin repeat module”
KDWLGWTPLHVAASHGHLEIVEVLLKAGADVNA

SEQ ID NO: 48 - protein, 33 aa, Artificial Sequence
  source: /note=“Description of Artificial Sequence Synthetic polypeptide”
  source: /note=“Ankyrin repeat module”
KDLSGRTPLHIAARQGHLEIVEVLLKAGADVNA

SEQ ID NO: 49 - protein, 33 aa, Artificial Sequence
  source: /note=“Description of Artificial Sequence Synthetic polypeptide”
  source: /note=“Ankyrin repeat module”
KDHYGWTPLHLAASEGHLEIVEVLLKAGADVNA

SEQ ID NO: 50 - protein, 33 aa, Artificial Sequence
  source: /note=“Description of Artificial Sequence Synthetic polypeptide”
  source: /note=“Ankyrin repeat module”
KDWIGWTPLHLAASFGHLEIVEVLLKAGADVNA

SEQ ID NO: 51 - protein, 33 aa, Artificial Sequence
  source: /note=“Description of Artificial Sequence Synthetic polypeptide”
  source: /note=“Ankyrin repeat module”
KDVSGKTPLHIAARQGHLEIVEVLLKAGADVNA

SEQ ID NO: 52 - protein, 33 aa, Artificial Sequence
  source: /note=“Description of Artificial Sequence Synthetic polypeptide”
  source: /note=“Ankyrin repeat module”
  MOD_RES (1)..(1): Any amino acid
  MOD_RES (3)..(4): Any amino acid
  MOD_RES (6)..(6): Any amino acid
  MOD_RES (13)..(15): Any amino acid
  MOD_RES (17)..(19): Any amino acid
  MOD_RES (22)..(22): Any amino acid
  MOD_RES (26)..(27): Any amino acid
XDXXGXTPLHLAXXXGXXXLVXVLLXXGADVNA

SEQ ID NO: 53 - protein, 33 aa, Artificial Sequence
  source: /note=“Description of Artificial Sequence Synthetic polypeptide”
  source: /note=“Ankyrin repeat module”
  MOD_RES (1)..(1): Any amino acid
  MOD_RES (3)..(4): Any amino acid
  MOD_RES (6)..(6): Any amino acid
  MOD_RES (14)..(15): Any amino acid
XDXXGXTPLHLAAXXGHLEIVEVLLKZGADVNA

SEQ ID NO: 54 - protein, 421 aa, Artificial Sequence
  source: /note=“Description of Artificial Sequence Synthetic polypeptide”
  source: /note=“TCE (without a half-life extending moiety)”
DLGKKLLEAARAGQDDEVRILMANGADVNALDWLGHTPLHLAAYEGHLEIVEVLLKNGADVNAIDDNNGFTPLHLAAIDGHLEIVEVLLKNGADVNAQDKFGKTAFDISIDNGNEDLAEILQKAAGSPTPTPTTPTPTPTTPTPTPTGSDLGDKLLLAATSGQDDEVRILLAAGADVNAKDYDGDTPLHLAADEGHLEIVEVLLKAGADVNAKDYSGSTPLHAAAAYGHLEIVEVLLKAGADVNAQDVFGYTPADLAAYVGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 55 - protein, 569 aa, Artificial Sequence
  source: /note=“Description of Artificial Sequence Synthetic polypeptide”
  source: /note=“TCE (with a half-life extending moiety)”
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLEAARAGQDDEVRILMANGADVNALDWLGHTPLHLAAYEGHLEIVEVLLKNGADVNAIDDNNGFTPLHLAAIDGHLEIVEVLLKNGADVNAQDKFGKTAFDISIDNGNEDLAEILQKAAGSPTPTPTTPTPTPTTPTPTPTGSDLGDKLLLAATSGQDDEVRILLAAGADVNAKDYDGDTPLHLAADEGHLEIVEVLLKAGADVNAKDYSGSTPLHAAAAYGHLEIVEVLLKAGADVNAQDVFGYTPADLAAYVGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

(19) United States
(12) Patent Application Publication
(10) Pub. No.: US 2024/0108746 A1
(43) Pub. Date: Apr. 4, 2024

(54) NOVEL SLOW-RELEASE PRODRUGS

(71) Applicant: MOLECULAR PARTNERS AG, Zurich (CH)

(72) Inventors: Andreas BOSSHART, Zurich (CH); Julia AHLSKOG, Zurich (CH); Bernd SCHLERETH, Zurich (CH); Patrick AMSTUTZ, Zurich (CH); Simon FONTAINE, Zurich (CH)

(21) Appl. No.: 18/267,282

(22) PCT Filed: Dec. 16, 2021
(86) PCT No.: PCT/IB2021/061888

§ 371 Date: Jun. 14, 2023

Related U.S. Application Data
(60) Provisional application No. 63/126,356, filed on Dec. 16, 2020.
(60) Provisional application No. 63/182,394, filed on Apr. 30, 2021.

(51) Int. Cl. A61K 47/68; C07K 14/725; C07K 16/28

(52) U.S. Cl. CPC A61K 47/6849; C07K 14/7051; C07K 16/2809; A61K 2039/505; C07K 2317/76; C07K 2317/92

(57) ABSTRACT

The present invention relates to compositions comprising a binding moiety and a drug molecule, wherein said binding moiety reversibly binds to said drug molecule to form a prodrug complex that slowly releases the drug molecule upon administration in vivo. The present invention further relates to methods of forming such compositions and to methods of treatment using such compositions. Also described are binding moieties, nucleic acids encoding said binding moieties and methods of making these using host cells.

### CROSS-REFERENCE TO RELATED APPLICATIONS

The present application claims the benefit of priority to U.S. 63/126,356, filed on Dec. 16, 2020; EP20216705, filed on 22 Dec. 2020; and U.S. 63/182,394, filed on Apr. 30, 2021. The disclosures of these patent applications are incorporated herein for all purposes by reference in their entirety.

### SEQUENCE LISTING

The instant application contains a Sequence Listing which has been submitted electronically in ASCII format and is hereby incorporated by reference in its entirety. Said ASCII copy, created on Dec. 16, 2021, is named 13081_0027-00304_SL.txt and is 54,310 bytes in size.

### FIELD OF THE INVENTION

The present invention relates to compositions comprising a binding moiety and a drug molecule, wherein said binding moiety reversibly binds to said drug molecule to form a prodrug complex that slowly releases the drug molecule upon administration in vivo. The present invention further relates to methods of forming such compositions and to methods of treatment using such compositions. Also described are binding moieties, nucleic acids encoding said binding moieties and methods of making these using host cells.

### BACKGROUND OF THE INVENTION

It is obvious that viable drug candidates need to meet certain efficacy standards, but it is also important that drug candidates have an acceptable safety profile. Many drug molecules have some adverse off-target and/or on-target side effects, and some drug molecules may also cause adverse on-target effects due to exaggerated and adverse pharmacologic effects at the intended target of the drug molecule. For certain drug classes adverse effects cannot be avoided so they must be mitigated. There are several options for this. For example, nonsteroidal anti-inflammatory drugs (NSAIDs) can damage the lining of the stomach, so they are often co-administered with an agent to protect the stomach, such as the proton-pump inhibitor omeprazole. For other drug molecules, the adverse effects, or the risk thereof, can be mitigated using a particular dosage regime such that the drug is administered to the patient in multiple doses or continuously over a longer period of time. Examples of this may include taking a drug multiple times a day, or intravenous (IV) infusion of a drug.

While divided doses and IV drug infusions are recognised and acceptable ways of dosing medication, they are not without drawbacks. Dosage regimes that are onerous on the patient, such as those requiring medication multiple times over a relatively short time period, are associated with poor patient compliance and consequently worse treatment outcomes. While IV infusion methods are less likely to suffer from poor patient compliance, the patient must be under medical care. This is disruptive for patients and results in added strain on the healthcare system. Consequently, there is a need in the art for improved ways of mitigating adverse drug effects.

Particularly problematic adverse effects associated with the use of various drugs are the cytokine release syndrome (CRS) and hypercytokinemia, also known as a “cytokine storm”. For example, CRS or hypercytokinemia may occur after treatment with certain immunotherapies, such as monoclonal antibodies and CAR-T cells. Hypercytokinemia typically occurs rapidly after the first dose of a drug and is characterised by an uncontrolled and excessive release of cytokines in the body. While cytokine release is a critical part of normal immune function, release of too many cytokines into the blood too quickly can cause symptoms such as high fever, inflammation, severe fatigue, nausea, and sometimes even multiple organ failure and death. A clinical trial for the drug Theralizumab, intended for the treatment of B cell chronic lymphocytic leukaemia and rheumatoid arthritis, had to be abandoned after the participants developed severe hypercytokinemia. The onset of symptoms occurred within an hour of dosing, and all of the participants in the trial required urgent hospital care. CRS is also caused by a large, rapid release of cytokines into the blood from immune cells affected by the immunotherapy. Symptoms of CRS include fever, nausea, headache, rash, rapid heartbeat, low blood pressure, and trouble breathing. Sometimes, CRS may be severe or life threatening.

One class of drugs known to be associated with CRS are T-cell engagers (TCEs). TCEs are also associated with systemic endothelial activation and massive lymphocyte redistribution, as well as neurological toxicities, particularly following first dose administration (Velasquez, Blood, 2018, 131(1), 30-38). These toxicities often impact clinical trial design and dose escalation strategies, and have proven dose limiting due to severity, especially in patients with high disease burden. Pre-medication and/or active intervention may also be required, ultimately leading to complex clinical trial design.

Several strategies have been devised for clinical management of CRS associated with the administration of drugs like T-cell engagers. These include step-dosing (stepwise dose-escalation), pre-treatment with steroids (especially dexamethasone) or treatment with tocilizumab (anti-IL6 receptor antibody) (see e.g., Aldoss et al., Current Oncology Reports, 2019, 21:4). Pre-treatment with steroids delays the onset of treatment, which is not recommended for aggressive disease states, and use of steroids may be contraindicated in patients with high body mass index (BMI) and/or blood pressure. Treatment with tocilizumab to avoid CRS was approved by the FDA in 2017. However, the immunosuppressive effect of this drug can leave patients vulnerable to other infectious diseases.

Taken together, there remains a need for novel or improved approaches to avoiding, reducing or mitigating the adverse effects, or the risk thereof, of drug molecules used for the treatment of diseases, including cancer.

### SUMMARY OF THE INVENTION

This application seeks to provide a novel approach to avoiding or mitigating adverse effects, or the risk thereof, following administration of a drug molecule. The present invention provides a method of slow release of an active drug molecule into the body upon administration of a drug product. The method uses so-called “slow-release” compositions (also referred to herein as prodrug complexes) that release active drug molecules into the body over a prolonged period of time, thereby avoiding a peak in active drug molecule concentration in the body shortly after administration. An example of a beneficial application of this method is the reduction of the risk of CRS following administration of TCEs, using slow-release compositions comprising a TCE.

Prodrug strategies are often adopted to mitigate problems associated with how a specific drug is absorbed, distributed, metabolized, and excreted. Many of the commonly available prodrugs contain small moieties that are hydrolysed in vivo (such as ester and amide groups), or groups that will be phosphorylated or dephosphorylated after administration.

In the present application, we describe a novel prodrug approach using binding moieties, which reversibly bind to a drug molecule and, when bound, inhibit a biological activity of the drug molecule. Such biological activity of the drug molecule may be, for example, the binding of the drug molecule to a biological target. Prodrug complexes of the invention comprise such a binding moiety reversibly bound to a drug molecule. The binding properties of the binding moiety to the drug molecule allow the release of the drug molecule as a function of time. Such release of the drug molecule as a function of time may occur, for example, upon administration of a prodrug complex of the invention to a subject, including a human. Binding moieties of the invention have a high affinity to and/or low off-rate from the drug molecule. The binding moieties of the invention include molecules with different structures, such as immunoglobulin molecules or non-immunoglobulin molecules. The binding moieties include antibodies, alternative scaffolds, such as engineered scaffolds, and polypeptides. An example of such an engineered scaffold is a designed ankyrin repeat domain.

Taken together, the present invention provides slow-release compositions comprising a binding moiety and a drug molecule, methods of making such compositions and methods of treatment using such compositions. The present invention further provides novel binding moieties, nucleic acids encoding said binding moieties and methods of making these using host cells.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
1. In a first embodiment, the present invention relates to a composition comprising (i) a binding moiety and (ii) a drug molecule; wherein said binding moiety reversibly binds to said drug molecule; and wherein said binding moiety, when bound, inhibits a biological activity of said drug molecule.
2. In a second embodiment, the present invention relates to the composition of embodiment 1, wherein said binding moiety comprises an antibody, an alternative scaffold, or a polypeptide.
3. In a third embodiment, the present invention relates to the composition of embodiments 1 or 2, wherein said binding moiety comprises an immunoglobulin molecule or a fragment thereof.
4. In a fourth embodiment, the present invention relates to the composition of embodiments 1 or 2, wherein said binding moiety comprises a non-immunoglobulin molecule.
5. In a fifth embodiment, the present invention relates to the composition of any of embodiments 1 to 4, wherein said binding moiety comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).
6. In a sixth embodiment, the present invention relates to the composition of any of embodiments 1 to 4, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).
6a. In embodiment 6a, the present invention relates to the composition of any of embodiments 1 to 4, wherein said binding moiety comprises an antigen binding domain that is derived from or is related to an adnectin.
6b. In embodiment 6b, the present invention relates to the composition of any of embodiments 1 to 4, wherein said binding moiety comprises an antigen binding domain that is derived from or is related to a monobody.
6c. In embodiment 6c, the present invention relates to the composition of any of embodiments 1 to 4, wherein said binding moiety comprises an antigen binding domain that is derived from or is related to an affibody.
6d. In embodiment 6d, the present invention relates to the composition of any of embodiments 1 to 4, wherein said binding moiety comprises an antigen binding domain that is derived from or is related to an affilin.
6e. In embodiment 6e, the present invention relates to the composition of any of embodiments 1 to 4, wherein said binding moiety comprises an antigen binding domain that is derived from or is related to an affimer.
6f. In embodiment 6f, the present invention relates to the composition of any of embodiments 1 to 4, wherein said binding moiety comprises an antigen binding domain that is derived from or is related to an aptamer.
6g. In embodiment 6g, the present invention relates to the composition of any of embodiments 1 to 4, wherein said binding moiety comprises an antigen binding domain that is derived from or is related to an affitin.
6h. In embodiment 6h, the present invention relates to the composition of any of embodiments 1 to 4, wherein said binding moiety comprises an antigen binding domain that is derived from or is related to an alphabody.
6i. In embodiment 6i, the present invention relates to the composition of any of embodiments 1 to 4, wherein said binding moiety comprises an antigen binding domain that is derived from or is related to a repeat protein domain.
6j. In embodiment 6j, the present invention relates to the composition of any of embodiments 1 to 4, wherein said binding moiety comprises an antigen binding domain that is derived from or is related to an armadillo repeat domain.
6k. In embodiment 6k, the present invention relates to the composition of any of embodiments 1 to 4, wherein said binding moiety comprises an antigen binding domain that is derived from or is related to an atrimer.
6l. In embodiment 6l, the present invention relates to the composition of any of embodiments 1 to 4, wherein said binding moiety comprises an antigen binding domain that is derived from or is related to an avimer.
6m. In embodiment 6m, the present invention relates to the composition of any of embodiments 1 to 4, wherein said binding moiety comprises an antigen binding domain that is derived from or is related to an ankyrin repeat domain.
6n. In embodiment 6n, the present invention relates to the composition of any of embodiments 1 to 4, wherein said binding moiety comprises an antigen binding domain that is derived from or is related to a fynomer.
6o. In embodiment 6o, the present invention relates to the composition of any of embodiments 1 to 4, wherein said binding moiety comprises an antigen binding domain that is derived from or is related to a knottin.
6p. In embodiment 6p, the present invention relates to the composition of any of embodiments 1 to 4, wherein said binding moiety comprises an antigen binding domain that is derived from or is related to a Kunitz domain.
6q. In embodiment 6q, the present invention relates to the composition of any of embodiments 1 to 4, wherein said binding moiety comprises an antigen binding domain that is derived from or is related to a T cell receptor (TCR).
7. In a seventh embodiment, the present invention relates to the composition of any preceding embodiment, wherein said biological activity of said drug molecule is binding of said drug molecule to a biological target.
8. In an eighth embodiment, the present invention relates to the composition of any preceding embodiment, wherein said biological activity of said drug molecule is an enzymatic activity.
9. In a ninth embodiment, the present invention relates to the composition of any preceding embodiment, wherein the binding affinity of said binding moiety to said drug molecule allows release of the drug molecule as a function of time upon administration of said composition to a mammal.
10. In a tenth embodiment, the present invention relates to the composition of embodiment 9, wherein said mammal is a human.
11. In an eleventh embodiment, the present invention relates to the composition of any preceding embodiment, wherein said binding moiety binds said drug molecule with a dissociation constant (K_{D}) of less than 10 nM, such as less than 10 nM, less than 1 nM, less than 100 pM, less than 10 pM or less than 1 pM.
12. In a twelfth embodiment, the present invention relates to the composition of any preceding embodiment, wherein said binding moiety has an off rate (k_{off}) from said drug molecule between about 1×10⁻⁸ s⁻¹ and about 1×10⁻⁴ s⁻¹, between about 1×10⁻⁸ s⁻¹ and about 1×10⁻⁵ s⁻¹, between about 1×10⁻⁸ s⁻¹ and about 1×10⁻⁶ s⁻¹, between about 1×10⁻⁸ s⁻¹ and about 1×10⁻⁷ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹ or between about 1×10⁻⁶ s⁻¹ and about 1×10⁻⁵ s⁻¹.
13. In a thirteenth embodiment, the present invention relates to the composition of embodiments 11 or 12, wherein said dissociation constant (K_{D}) or off rate (k_{off}) is measured in phosphate buffered saline (PBS).
14. In a fourteenth embodiment, the present invention relates to the composition of any preceding embodiment, wherein said binding moiety has a blocking half-life (T_{1/2}) when complexed with said drug molecule, wherein said blocking half-life is calculated according to the following formula:

$$blocking {T}_{1 / 2} = \frac{ln ( 2 )}{{k}_{off}} .$$

15. In a fifteenth embodiment, the present invention relates to the composition of embodiment 14, wherein said blocking half-life (T_{1/2}) is at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, such as at least about 20 hours, at least about 25 hours, at least about 30 hours, at least about 35 hours, at least about 40 hours, at least about 45 hours, at least about 50 hours, at least about 55 hours or at least about 60 hours.
16. In a sixteenth embodiment, the present invention relates to the composition of any preceding embodiment, wherein said binding moiety comprises a designed ankyrin repeat domain.
17. In a seventeenth embodiment, the present invention relates to the composition of embodiment 16, wherein said designed ankyrin repeat domain comprises an ankyrin repeat module comprising an amino acid sequence selected from the group consisting of (1) SEQ ID NOs: 30 to 51 and (2) sequences in which up to 9 amino acids in any of SEQ ID NOs: 30 to 51 are substituted by another amino acid.
18. In an eighteenth embodiment, the present invention relates to the composition of embodiment 16, wherein said designed ankyrin repeat domain comprises an amino acid sequence selected from the group consisting of (1) SEQ ID NOs: 1 to 10 and (2) sequences that have at least 85% amino acid sequence identity with any of SEQ ID NOs: 1 to 10.
19. In a nineteenth embodiment, the present invention relates to the composition of any preceding embodiment, wherein said drug molecule comprises an antibody, an alternative scaffold, or a polypeptide.
20. In a twentieth embodiment, the present invention relates to the composition of any preceding embodiment, wherein said drug molecule comprises an immunoglobulin molecule or a fragment thereof.
21. In a twenty first embodiment, the present invention relates to the composition of any preceding embodiment, wherein said drug molecule comprises a non-immunoglobulin molecule.
22. In a twenty second embodiment, the present invention relates to the composition of any preceding embodiment, wherein said drug molecule comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).
23. In a twenty third embodiment, the present invention relates to the composition of any preceding embodiment, wherein said drug molecule comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).
23a. In embodiment 23a, the present invention relates to the composition of any preceding embodiment, wherein said binding moiety comprises an antigen binding domain that is derived from or is related to an adnectin.
23b. In embodiment 23b, the present invention relates to the composition of any preceding embodiment, wherein said binding moiety comprises an antigen binding domain that is derived from or is related to a monobody.
23c. In embodiment 23c, the present invention relates to the composition of any preceding embodiment, wherein said binding moiety comprises an antigen binding domain that is derived from or is related to an affibody.
23d. In embodiment 23d, the present invention relates to the composition of any preceding embodiment, wherein said binding moiety comprises an antigen binding domain that is derived from or is related to an affilin.
23e. In embodiment 23e, the present invention relates to the composition of any preceding embodiment, wherein said binding moiety comprises an antigen binding domain that is derived from or is related to an affimer.
23f. In embodiment 23f, the present invention relates to the composition of any preceding embodiment, wherein said binding moiety comprises an antigen binding domain that is derived from or is related to an aptamer.
23g. In embodiment 23g, the present invention relates to the composition of any preceding embodiment, wherein said binding moiety comprises an antigen binding domain that is derived from or is related to an affitin.
23h. In embodiment 23h, the present invention relates to the composition of any preceding embodiment, wherein said binding moiety comprises an antigen binding domain that is derived from or is related to an alphabody.
23i. In embodiment 23i, the present invention relates to the composition of any preceding embodiment, wherein said binding moiety comprises an antigen binding domain that is derived from or is related to a repeat protein domain.

23j. In embodiment 23j, the present invention relates to the composition of any preceding embodiment, wherein said binding moiety comprises an antigen binding domain that is derived from or is related to an armadillo repeat domain.
23k. In embodiment 23k, the present invention relates to the composition of any preceding embodiment, wherein said binding moiety comprises an antigen binding domain that is derived from or is related to an atrimer.
23l. In embodiment 23l, the present invention relates to the composition of any preceding embodiment, wherein said binding moiety comprises an antigen binding domain that is derived from or is related to an avimer.
23m. In embodiment 23m, the present invention relates to the composition of any preceding embodiment, wherein said binding moiety comprises an antigen binding domain that is derived from or is related to an ankyrin repeat domain.
23n. In embodiment 23n, the present invention relates to the composition of any preceding embodiment, wherein said binding moiety comprises an antigen binding domain that is derived from or is related to a fynomer.
23o. In embodiment 23o, the present invention relates to the composition of any preceding embodiment, wherein said binding moiety comprises an antigen binding domain that is derived from or is related to a knottin.
23p. In embodiment 23p, the present invention relates to the composition of any preceding embodiment, wherein said binding moiety comprises an antigen binding domain that is derived from or is related to a Kunitz domain.
23q. In embodiment 23q, the present invention relates to the composition of any preceding embodiment, wherein said binding moiety comprises an antigen binding domain that is derived from or is related to a T cell receptor (TCR).
24. In a twenty fourth embodiment, the present invention relates to the composition of any preceding embodiment, wherein said drug molecule has binding specificity for CD3.
25. In a twenty fifth embodiment, the present invention relates to the composition of any preceding embodiment, wherein said drug molecule further comprises at least one binding domain with binding specificity for a tumor-associated antigen (TAA).
25a. In embodiment 25a, the present invention relates to the composition of embodiment 25, wherein said tumor-associated antigen (TAA) is CD33.
25b. In embodiment 25b, the present invention relates to the composition of embodiment 25, wherein said tumor-associated antigen (TAA) is CD123.
26. In a twenty sixth embodiment, the present invention relates to the composition of any preceding embodiment, wherein said drug molecule is a T-cell engager drug molecule (TCE).
27. In a twenty seventh embodiment, the present invention relates to the composition of any preceding embodiment, wherein said drug molecule comprises a designed ankyrin repeat domain.
28. In a twenty eighth embodiment, the present invention relates to the composition of embodiment 27, wherein said designed ankyrin repeat domain comprises an amino acid sequence selected from the group consisting of (1) SEQ ID NOs: 12 to 15 and (2) sequences that have at least 85% amino acid sequence identity with any of SEQ ID NOs: 12 to 15.
29. In a twenty ninth embodiment, the present invention relates to the composition of any preceding embodiment, wherein said drug molecule comprises an antibody.
30. In a thirtieth embodiment, the present invention relates to the composition of embodiment 29, wherein said drug molecule is an antibody that is a T-cell engager drug molecule (TCE).
31. In a thirty first embodiment, the present invention relates to the composition of embodiment 30, wherein said TCE comprises a binding domain which binds to CD3 and further comprises a binding domain which binds a tumor-associated antigen (TAA).
31a. In embodiment 31a, the present invention relates to the composition of embodiment 31, wherein said tumor-associated antigen (TAA) is CD33.
31 b. In embodiment 31 b, the present invention relates to the composition of embodiment 31, wherein said tumor-associated antigen (TAA) is CD123.
32. In a thirty second embodiment, the present invention relates to the composition of any of embodiments 24 to 31 b, wherein binding of said binding moiety to said TCE drug molecule inhibits binding of said TCE drug molecule to T cells and/or activation of T cells.
33. In a thirty third embodiment, the present invention relates to the composition of any one of embodiments 24 to 28 and 30 to 32, wherein said TCE is a bispecific antibody.
34. In a thirty fourth embodiment, the present invention relates to the composition of any preceding embodiment, wherein said binding moiety is an anti-idiotypic binder of said drug molecule.
35. In a thirty fifth embodiment, the present invention relates to the composition of any preceding embodiment additionally comprising a pharmaceutically acceptable carrier or excipient.
36. In a thirty sixth embodiment, the present invention relates to the composition of any preceding embodiment for use in therapy.
37. In a thirty seventh embodiment, the present invention relates to the composition for use according to embodiment 36, for use in treating a proliferative disease, optionally wherein said proliferative disease is cancer.
38. In a thirty eighth embodiment, the present invention relates to a method of treatment comprising the step of administering to a subject in need thereof a composition as defined in any one of embodiments 1 to 35.
39. In a thirty ninth embodiment, the present invention relates to the method of embodiment 38, wherein said method is a method of treating a proliferative disease, optionally wherein said proliferative disease is cancer.
40. In a fortieth embodiment, the present invention relates to a method of T cell activation in a subject in need thereof, the method comprising the step of administering to said subject the composition of any one of embodiments 24 to 28 and 30 to 32, optionally wherein said composition additionally comprises a pharmaceutically acceptable carrier or excipient.
41. In a forty first embodiment, the present invention relates to a method of controlling release of an active drug molecule in vivo comprising administering the composition of any one of embodiments 1 to 35 to a subject in need thereof.
42. In a forty second embodiment, the present invention relates to the method of any one of embodiments 38 to 41, wherein said subject is a human.
43. In a forty third embodiment, the present invention relates to a method of making a controlled release formulation comprising the steps of:
   (i) providing a binding moiety as defined in any one of embodiments 1 to 6, 9 and 11 to 18;
   (ii) providing a drug molecule as defined in any one of embodiments 19 to 23; and
   (iii) allowing said binding moiety and active drug molecule to reach an equilibrium such that substantially all of said drug molecule is bound by said binding moiety.
44. In a forty fourth embodiment, the present invention relates to a method of controlling the biological activity of a drug molecule, the method comprising combining a binding moiety as defined in any one of embodiments 1 to 6, 9 and 11 to 18 with a drug molecule as defined in any one of embodiments 19 to 23 to form a composition, and administering said composition to a patient in need thereof.
45. In a forty fifth embodiment, the present invention relates to the method of embodiment 44, wherein said biological activity of said drug molecule is binding of said drug molecule to a biological target.
46. In a forty sixth embodiment, the present invention relates to the method of embodiment 45, wherein said biological activity of said drug molecule is an enzymatic activity.
47. In a forty seventh embodiment, the present invention relates to a binding moiety having binding specificity for a drug molecule, wherein said binding moiety, when bound to said drug molecule, inhibits a biological activity of said drug molecule.
48. In a forty eighth embodiment, the present invention relates to the binding moiety of embodiment 47, wherein binding of said binding moiety to said drug molecule forms a complex that reversibly inhibits a biological activity of said drug molecule.
49. In a forty ninth embodiment, the present invention relates to the binding moiety of any of embodiments 47 or 48, wherein said biological activity of said drug molecule is binding of said drug molecule to a biological target.
50. In a fiftieth embodiment, the present invention relates to the binding moiety of any of embodiments 47 to 49, wherein said biological activity of said drug molecule is an enzymatic activity.
51. In a fifty first embodiment, the present invention relates to the binding moiety of any one of embodiments 47 to 50, having a binding affinity (K_{D}) to said drug molecule of less than 10 nM, such as less than 10 nM, less than 1 nM, less than 100 pM, less than 10 pM or less than 1 pM.
52. In a fifty second embodiment, the present invention relates to the binding moiety of any one of embodiments 47 to 51, wherein said binding moiety has an off rate (k_{off}) from said drug molecule between about 1×10⁻⁸ s⁻¹ and about 1×10⁻⁴ s⁻¹, between about 1×10⁻⁸ s⁻¹ and about 1×10⁻⁵ s⁻¹, between about 1×10⁻⁸ s⁻¹ and about 1×10⁻⁶ s⁻¹, between about 1×10⁻⁸ s⁻¹ and about 1×10⁻⁷ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹ or between about 1×10⁻⁶ s⁻¹ and about 1×10⁻⁵ s⁻¹.
53. In a fifty third embodiment, the present invention relates to the binding moiety of embodiments 51 or 52, wherein said dissociation constant (K_{D}) or off rate (k_{off}) is measured in phosphate buffered saline (PBS).
54. In a fifty fourth embodiment, the present invention relates to the binding moiety of any one of embodiments 47 to 53, wherein said binding moiety has a blocking half-life (T_{1/2}) when complexed with said drug molecule, and wherein said blocking half-life is calculated according to the following formula:

$$blocking {T}_{1 / 2} = \frac{ln ( 2 )}{{k}_{o f f}} .$$

55. In a fifty fifth embodiment, the present invention relates to the binding moiety of embodiment 54, wherein said blocking half-life (T_{1/2}) is at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, such as at least about 20 hours, at least about 25 hours, at least about 30 hours, at least about 35 hours, at least about 40 hours, at least about 45 hours, at least about 50 hours, at least about 55 hours or at least about 60 hours.
56. In a fifty sixth embodiment, the present invention relates to the binding moiety of any one of embodiments 47 to 55, wherein said binding moiety comprises an antibody, an alternative scaffold, or a polypeptide.
57. In a fifty seventh embodiment, the present invention relates to the binding moiety of any one of embodiments 47 to 56, wherein said binding moiety comprises an immunoglobulin molecule or a fragment thereof.
58. In a fifty eighth embodiment, the present invention relates to the binding moiety of any one of embodiments 47 to 56, wherein said binding moiety comprises a non-immunoglobulin molecule.
59. In a fifty ninth embodiment, the present invention relates to the binding moiety of any one of embodiments 47 to 58, wherein said binding moiety comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).
60. In a sixtieth embodiment, the present invention relates to the binding moiety of any one of embodiments 47 to 59, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).
60a. In embodiment 60a, the present invention relates to the composition of any of embodiments 47 to 59, wherein said binding moiety comprises an antigen binding domain that is derived from or is related to an adnectin.
60b. In embodiment 60b, the present invention relates to the composition of any of embodiments 47 to 59, wherein said binding moiety comprises an antigen binding domain that is derived from or is related to a monobody.
60c. In embodiment 60c, the present invention relates to the composition of any of embodiments 47 to 59, wherein said binding moiety comprises an antigen binding domain that is derived from or is related to an affibody.
60d. In embodiment 60d, the present invention relates to the composition of any of embodiments 47 to 59, wherein said binding moiety comprises an antigen binding domain that is derived from or is related to an affilin.
60e. In embodiment 60e, the present invention relates to the composition of any of embodiments 47 to 59, wherein said binding moiety comprises an antigen binding domain that is derived from or is related to an affimer.
60f. In embodiment 60f, the present invention relates to the composition of any of embodiments 47 to 59, wherein said binding moiety comprises an antigen binding domain that is derived from or is related to an aptamer.
60g. In embodiment 60g, the present invention relates to the composition of any of embodiments 47 to 59, wherein said binding moiety comprises an antigen binding domain that is derived from or is related to an affitin.
60h. In embodiment 60h, the present invention relates to the composition of any of embodiments 47 to 59, wherein said binding moiety comprises an antigen binding domain that is derived from or is related to an alphabody.
60i. In embodiment 60i, the present invention relates to the composition of any of embodiments 47 to 59, wherein said binding moiety comprises an antigen binding domain that is derived from or is related to a repeat protein domain.
60j. In embodiment 60j, the present invention relates to the composition of any of embodiments 47 to 59, wherein said binding moiety comprises an antigen binding domain that is derived from or is related to an armadillo repeat domain.
60k. In embodiment 60k, the present invention relates to the composition of any of embodiments 47 to 59, wherein said binding moiety comprises an antigen binding domain that is derived from or is related to an atrimer.
60l. In embodiment 60l, the present invention relates to the composition of any of embodiments 47 to 59, wherein said binding moiety comprises an antigen binding domain that is derived from or is related to an avimer.
60m. In embodiment 60m, the present invention relates to the composition of any of embodiments 47 to 59, wherein said binding moiety comprises an antigen binding domain that is derived from or is related to an ankyrin repeat domain.
60n. In embodiment 60n, the present invention relates to the composition of any of embodiments 47 to 59, wherein said binding moiety comprises an antigen binding domain that is derived from or is related to a fynomer.
60o. In embodiment 60o, the present invention relates to the composition of any of embodiments 47 to 59, wherein said binding moiety comprises an antigen binding domain that is derived from or is related to a knottin.
60p. In embodiment 60p, the present invention relates to the composition of any of embodiments 47 to 59, wherein said binding moiety comprises an antigen binding domain that is derived from or is related to a Kunitz domain.
60q. In embodiment 60q, the present invention relates to the composition of any of embodiments 47 to 59, wherein said binding moiety comprises an antigen binding domain that is derived from or is related to a T cell receptor (TCR).
61. In a sixty first embodiment, the present invention relates to the binding moiety of any one of embodiments 47 to 60, wherein said binding moiety is a designed ankyrin repeat domain.
62. In a sixty second embodiment, the present invention relates to the binding moiety of embodiment 61, wherein said designed ankyrin repeat domain comprises an ankyrin repeat module comprising an amino acid sequence selected from the group consisting of (1) SEQ ID NOs: 30 to 51 and (2) sequences in which up to 9 amino acids in any of SEQ ID NOs: 30 to 51 are substituted by another amino acid.
63. In a sixty third embodiment, the present invention relates to the binding moiety of any of embodiments 61 or 62, wherein said designed ankyrin repeat domain comprises an amino acid sequence selected from the group consisting of (1) SEQ ID NOs: 1 to 10 and (2) sequences that have at least 85% amino acid sequence identity with any of SEQ ID NOs: 1 to 10.
64. In a sixty fourth embodiment, the present invention relates to a nucleic acid encoding the binding moiety of any of embodiments 47 to 60.
65. In a sixty fifth embodiment, the present invention relates to a nucleic acid encoding the designed ankyrin repeat domain of any of embodiments 61 to 63.
66. In a sixty sixth embodiment, the present invention relates to a host cell comprising the nucleic acid molecule of embodiments 64 or 65.
67. In a sixty seventh embodiment, the present invention relates to a method of making the binding moiety according to any one of embodiments 47 to 63, comprising culturing the host cell of embodiment 66 under conditions wherein said binding moiety is expressed.
68. In a sixty eighth embodiment, the present invention relates to the method of embodiment 67, wherein said host cell is a prokaryotic host cell.
69. In a sixty ninth embodiment, the present invention relates to the method of embodiment 67, wherein said host cell is a eukaryotic host cell.

### BRIEF DESCRIPTION OF THE FIGURES

FIG. 1: A computer simulation of the concentrations of total drug molecule (“Total drug”), prodrug complex (“Complexed drug”), unbound drug molecule (“Free drug”), and unbound binding moiety (“Free binder”) in the plasma, following a single dose administration of a prodrug complex of the invention to a patient. FIG. 1 illustrates the development of concentrations of the different molecules and prodrug complex over time (a period of 10 weeks), starting with substantially fully complexed drug molecule at the time of administration. The concentration of unbound (active) drug reaches a concentration higher than the complexed (inactive) drug after around 10 days and makes up the large majority of the total drug after 5 weeks. Free binder is rapidly cleared from the system and hence is not able to re-bind the drug molecule after dissociation from it. As illustrated, the concentration of free (active) drug never reaches the concentration of total drug at the time of administration. Hence, due to the slow release of drug molecule from the prodrug complex, the maximum concentration (Cₘₐₓ) of free (active) drug obtained after administration of a prodrug complex of the invention is significantly reduced as compared to the Cₘₐₓ obtained after administration of the same total amount of drug molecule in an uncomplexed form.

FIG. 2: A computer simulation of the concentration of unbound (active) drug molecule following repeated dose administration of either unbound drug molecule (“Free drug without binder”, dashed line) or prodrug complexes comprising the drug molecule bound to a binding moiety (“Drug+binder”, solid lines), whereby in each case the same total amount of drug molecule is administered to a patient. Three different prodrug complexes are shown, generated with binding moieties with different binding affinities for the drug molecule, as indicated. FIG. 2 shows that reduced Cₘₐₓ and a slowed increase in exposure of active drug molecule can be achieved with the prodrug complexes of the invention. Furthermore, the extent of Cₘₐₓ reduction and slowing of increase in exposure depends on the binding affinity of the binding moiety to the drug molecule. The higher the binding affinity, the stronger the reduction of Cₘₐₓ and the slower the increase in exposure.

FIG. 3a: Before administration of a prodrug complex to a patient, the binding moiety (“Binder”) binds tightly to the drug molecule (“Drug”), thereby forming the prodrug complex. Since in the prodrug formulation the concentrations of both binding moiety and drug are high, kₒₙ dominates the binding equilibrium and substantially all of the drug molecules are bound by binding moiety. If bound by the binding moiety, the biological activity of the drug molecule is inhibited.

FIG. 3b: Following administration to a patient, the concentrations of both binding moiety and drug in solution are greatly reduced, and the equilibrium shifts to favour dissociation of the prodrug complex (i.e., koff dominates the binding equilibrium). The drug molecules are therefore de-complexed from the binding moiety over time, slowly releasing active drug molecules into the body of the patient.

FIG. 4: Surface plasmon resonance (SPR) data of different binding moieties (Binder #1, #4 and #5), tested alongside a precursor of Binder #9, and the parental binder as a reference, showing that the parental binder has a blocking T_{1/2} of 1-2 hours, whereas the binding moieties tested in Example 2 show much less dissociation after the 2 h measurement period. This much reduced dissociation results in much longer blocking T_{1/2} as compared to the parental binder.

FIGS. 5a and 5b: Standard tumor cell killing and T-cell activation assay with TCE #1 and 3 different binding moieties (Binder #4, #5 and #9) and the mid-affinity parental binder. Sample preparation was as described in Example 3. Tumor cell killing (determined by lactate dehydrogenase (LDH) release, OD₄₉₂-OD₆₂₀) is shown in FIG. 5a and T-cell activation (CD25+ cells as % of CD8+ cells) is shown in FIG. 5b. IC50 values in nM are as indicated.

FIG. 6a: T-cell mediated growth inhibition (used as a surrogate of T-cell mediated killing) of TAA-expressing tumor cells (labelled with NucLight Red®) in the presence of serially diluted TCE (TCE #1) was determined by Incucyte®; see Example 4. Total red object area (corresponding to tumor cells) was determined at regular intervals. Error bars show standard error per image (4 images per well).

FIG. 6b: Plot of AUC (area under curve) data against the concentration of TCE #1 from Incucyte® experiment until timepoint 4.5 days. EC50 values are indicated in pM; see Example 4.

FIGS. 7a to 7e: “Simple” Incucyte® experiment to compare a super-high affinity and a mid-affinity binding moiety for their ability to block T-cell mediated growth inhibition (used as a surrogate of T-cell mediated killing) of TAA-expressing tumor cells over time, as described in Example 4. The following conditions were tested: no addition of TCE or binding moiety (Background); addition of only 10 nM binding moiety (Binder #4, #5 and #9; FIG. 7e); addition of only TCE (TCE #1); addition of pre-equilibrated TCE #1 and parental binder at decreasing ratios of binding moiety:TCE (TCE #1+parental Binder); addition of pre-equilibrated TCE and super-high affinity binding moiety (Binder #5) at decreasing ratios of binding moiety:TCE (TCE #1+Binder #5). TCE concentration was set to 10 pM final concentration, corresponding roughly to EC₈₀ of TCE #1 based on previous LDH experiments. Total red object area (corresponding to tumor cells) was determined at regular intervals. Error bars show standard error per image (4 images per well).

FIGS. 8a to 8d: “Simple” Incucyte® experiment to compare different super-high affinity binding moieties for their ability to block T-cell mediated growth inhibition (used as a surrogate of T-cell mediated killing) of TAA-expressing tumor cells over time. A constant concentration of TCE (TCE #1, 10 pM final concentration) was mixed with a titration of different binding moieties (Binder #4, #5 and #9). The mixtures were pre-incubated at 100× concentration (to allow >99.9% binding of TCE by binding moiety) for at least 24 h and then diluted 100-fold just before starting the Incucyte® experiment. Negative control curves for 10 nM binding moiety only (Binder #4, #5 and #9) are shown in FIG. 8d. Background curves were done with no addition of TCE or binding moiety. Total red object area (corresponding to tumor cells) was determined at regular intervals; see Example 4. Error bars show standard error per image (4 images per well).

FIGS. 9a to 9b: “Complex” Incucyte® experiment to compare the ability of a super-high affinity binding moiety to block T-cell mediated growth inhibition (used as a surrogate of T-cell mediated killing) of TAA-expressing tumor cells overtime in the absence or presence of a functional sink compartment (see Example 6). The experiment was performed either with an inlet containing uncoated beads (i.e., in the absence of a functional sink compartment) (FIG. 9a) or with an inlet containing beads coated with a CD3 binding domain able to capture dissociated binder (i.e., in the presence of a functional sink compartment) (FIG. 9b). T-cell mediated growth inhibition curves of TAA-expressing tumor cells in the presence of T-cells and prodrug complexes prepared with different molar ratios of TCE #1 to Binder #9 are shown in FIG. 9a for the setup with uncoated beads, and in FIG. 9b for the setup with coated beads. Controls were done with unbound TCE #1 (instead of a prodrug complex), and without any TCE or prodrug complex (Background), as indicated. Total red object area (corresponding to tumor cells) was determined at regular intervals. Error bars show standard error per image (36 images per well).

FIGS. 10a to 10f: Cytokine release determined in an ex vivo assay by spiking control or drug molecules into fresh human whole blood containing CD123+ and CD33+ target cells and incubating samples on a rotating wheel to avoid blood clotting. Tested control and drug molecules included vehicle (stars), 1 nM α-CD123×α-CD3 industry control (filled triangles), and 1 nM TCE #1 (empty squares), or in comparison thereto 1 nM TCE #1+1.2 nM Binder #5 (empty diamonds). (A) and (B) show the TCE drug molecule in the unbound form (A) and the complexed form with Binder #5 (B). Cytokine release for TNF-α (C), IFN-γ (D), IL-2 (E) and IL-6 (F) was determined at 0 h, 2 h, 4 h, 8 h, 24 h by Meso Scale MULTI-ARRAY® technology. Data is from a single experiment performed at Immuneed AB, Sweden with blood from one healthy human donor.

FIGS. 11a to 11g: In vivo efficacy study comparing anti-tumor activity of A) unbound TCE to B) TCE complexed with two different Binders in Molm-13 tumor-bearing, PBMC engrafted NOG mice. C) Study design of in vivo efficacy study. PBMCs from two human donors were engrafted on d0, Molm-13 tumor cells were injected s.c. on d2 and treatment was started on d6 at tumor size ˜70 mm³. Unbound TCE #2 given at two different doses 200 μg/kg and 1000 μg/kg was compared to 1000 μg/kg TCE #2 in complex with two different Binders having different affinities to the CD3 binding domain. Complexes were pre-equilibrated with a 2-fold molar excess of Binder to guarantee 100% complexation rate of the TCE at treatment start. Treatment was given i.v. 3qw for all treatment groups including vehicle, except for the non-half-life-extended α-CD33×α-CD3 industry control (group 2), where treatment was given daily at 200 μg/kg. Blood samples were taken prior and 4 h post first treatment dose in order to determine cytokine levels at treatment start with these relatively small tumors. D) Tumor growth curves for all six treatment groups plotted as mean±SEM for n=10 mice humanized either with PBMCs of Donor A or B, except for group 2 (industry control) where two animals were excluded due to non-successful humanization for Donor B. Treatment period starting on d6 and ending on d16 is indicated. E) Tumor growth curves for all six treatment groups plotted as mean±SEM for n=5 mice humanized with PBMCs of Donor A. F) Tumor growth curves for all six treatment groups plotted as mean±SEM for n=5 mice humanized with PBMCs of Donor B, except for group 2 where two animals were excluded due to non-successful humanization. G) Legend of all six treatment groups.

FIGS. 12a to 12d: Human cytokine levels measured in serum from blood samples taken prior (pre) or 4 h post first dose during the in vivo efficacy study with tumors being relatively small at treatment start (˜70 mm³). Cytokine levels of the individual animals are plotted as filled symbols (Donor A, n=5) or empty symbols (Donor B, n=5) for the six treatment groups introduced in FIG. 11 with the mean being displayed as bar±SD. As in FIG. 11 two mice of treatment group 2 had to be excluded due to non-successful humanization for Donor B. A) TNF-α levels in pg/ml. B) IFN-γ levels in pg/ml. C) IL-2 levels in pg/ml and D) IL-6 levels in pg/ml.

FIGS. 13a to 13d: Independent in vivo safety studies comparing cytokine release of A) unbound TCE to B) TCE complexed with two different Binders in Molm-13 tumor-bearing, PBMC engrafted NOG mice. C) Study design of in vivo safety study. PBMCs from two human donors were engrafted on d0, MOLM-13 tumor cells were injected subcutaneously (s.c.) into the hind flank on d2 and a single dose (challenge) was injected intravenously (i.v.) on d14 at tumor size ˜300-800 mm³. Unbound TCE #2 given at 1000 μg/kg was compared to TCE #2 in complex with three different Binders having different affinities ranging from K_{D}≤1 pM to double-digit pM to the CD3 binding domain. Complexes were pre-equilibrated with a 2-fold molar excess of Binder to guarantee 100% complexation rate of the TCE at treatment start. Blood samples were taken pre-dose, 2 h, 4 h, 8 h and 24 h post first treatment dose in order to determine cytokine levels with mid-size tumors. D) Human cytokine levels (TNF-α, IFN-γ, IL-2 and IL-6) in serum from blood samples taken prior or post a single dose treatment in an in vivo safety study with established tumors (300-800 mm³). Cytokine levels were determined on undiluted serum samples by CBA human Th1/Th2/Th17 kit (BD Biosciences). Cytokine levels of the individual animals are plotted as filled symbols (Donor A, n=5) or empty symbols (Donor B, n=5), and mean values are displayed as solid black and solid grey lines, respectively, for the non-blocked TCE #2 vs. TCE #2+Binder treatment groups. In vivo safety study 1 was carried out using TCE #2±Binder #1 or Binder #4, while the separate but comparable in vivo safety study 2 focused on TCE #2±Binder #10.

### DETAILED DESCRIPTION OF THE INVENTION

### Overview

The present invention relates to compositions comprising a binding moiety and a drug molecule, wherein said binding moiety reversibly binds to the drug molecule, and wherein said binding moiety, when bound, inhibits a biological activity of the drug molecule. Such a composition of the invention is a prodrug complex that slowly releases the drug molecule upon administration in vivo. Slowed release of active drug molecule upon administration can minimise adverse effects, or the risk thereof, otherwise associated with the drug molecule.

Without wishing to be bound by theory, it is understood that the binding moieties described herein bind tightly in a non-covalent fashion to a drug molecule. Binding may take place at the active site of the drug molecule, or at another site on the drug molecule. Binding may be anti-idiotypic. Irrespective of the binding location, binding of the binding moiety inhibits the biological activity (i.e., mode of action) of the drug molecule. The drug molecule is formulated with an excess of the binding moiety, resulting in a complete or almost complete complexation and inhibition of the drug molecule, and formation of a composition (i.e., a prodrug complex). Upon administration to a patient, the prodrug complex is diluted strongly, leading to continuous release of active drug molecule over time, as re-binding of the binding moiety becomes negligible due to i) the very low concentration of binding moiety and drug in the body and ii) rapid elimination of the binding moiety from the system.

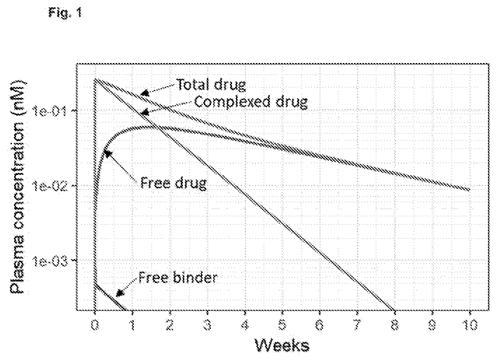

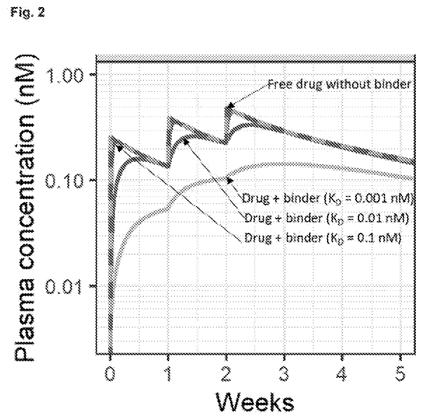

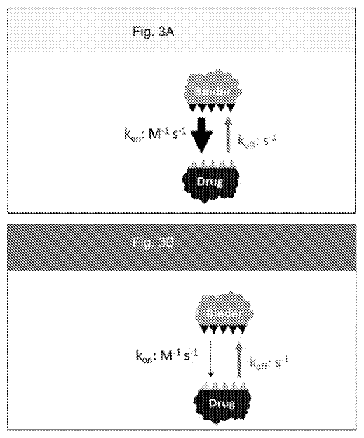

FIGS. 1 to 3 provide further illustration and explanation of the slow release concept and the properties of the binding moieties and compositions (prodrug complexes) of the invention.

The present invention further relates to methods of forming such compositions and to methods of treatment using such compositions, and to use of such compositions in therapy. For example, the present invention relates to use of such compositions in the treatment of proliferative diseases, such as cancer.

Also described are binding moieties having binding specificity for a drug molecule, wherein binding of said binding moiety to said drug molecule forms a complex that reversibly inhibits a biological activity of said drug molecule. For example, such biological activity of said drug molecule is binding of said drug molecule to a biological target.

The invention further relates to nucleic acids encoding said binding moieties and methods of making these using host cells.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g., by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “drug molecules” (used interchangeably herein with the term “drugs”) refers to therapeutic agents that comprise a polypeptide or a protein, wherein said polypeptide or protein contains a site that is capable of being bound by a binding moiety.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding moiety” or “binder” refers to a binding agent that comprises a polypeptide or a protein, wherein said polypeptide or protein is capable of non-covalently binding to a drug molecule. The binding moiety does not necessarily need to bind to an active site of the drug molecule. The binding moiety must, however, bind in such a way as to inhibit the mode of action of the drug. This may be by binding to the active site of the drug, but may also be by binding to another site on the drug molecule to either change the conformation of said drug (i.e., allosteric inhibition), or to sterically hinder the active site of the drug molecule. The active site of the drug molecule is involved in a biological activity of the drug molecule. The biological activity can be an enzymatic activity or binding to a biological target. Binding of a binding moiety to a drug molecule inhibits a biological activity of the drug molecule. For example, binding of a binding moiety to a drug molecule inhibits the enzymatic activity of the drug molecule or inhibits binding of the drug molecule to a biological target. Binding of a binding moiety to a drug molecule may be anti-idiotypic. Thus, a binding moiety may be an anti-idiotypic binder of a drug molecule.

The binding moieties used in the present invention include antibodies, alternative scaffolds, and polypeptides. As used herein, the term “antibody” refers not only to intact antibody molecules, such as those typically produced by the immune system when it detects foreign antigens, but also to any fragments, variants and synthetic or engineered analogues of antibody molecules that retain antigen-binding ability. Such fragments, variants and analogues are also well known in the art and are regularly employed in vitro or in vivo. Accordingly, the term “antibody” encompasses intact immunoglobulin molecules, antibody fragments such as, e.g., Fab, Fab′, F(ab′)₂, and single chain V region fragments (scFv), bispecific or multispecific antibodies, chimeric antibodies, humanized antibodies, antibody fusion proteins, unconventional antibodies, and proteins comprising an antigen binding domain derived from an immunoglobulin molecule. As used herein, the term “alternative scaffolds” refers to any molecule comprising or consisting of a protein, but that is not an antibody.

A binding moiety of any of these different structural types can bind tightly to a drug molecule, blocking the mode of action of the drug molecule and forming a prodrug complex. The prodrug complex slowly de-complexes in vivo, releasing the drug molecule into the body. In one preferred embodiment, a binding moiety comprises an ankyrin repeat domain with binding specificity for a drug molecule. In another preferred embodiment, a binding moiety comprises an antibody with binding specificity for a drug molecule. In another preferred embodiment, a binding moiety comprises an alternative scaffold with binding specificity for a drug molecule, wherein the alternative scaffold does not comprise an ankyrin repeat domain. In one preferred embodiment, the drug molecule comprises an antibody and the binding moiety is an anti-idiotypic antibody with binding specificity for said antibody comprised in said drug molecule. In another preferred embodiment, the drug molecule comprises an antibody and the binding moiety is an anti-idiotypic alternative scaffold, such as, e.g. an ankyrin repeat domain, with binding specificity for said antibody comprised in said drug molecule. In another preferred embodiment, the drug molecule comprises an alternative scaffold, such as, e.g. an ankyrin repeat domain, and the binding moiety is an anti-idiotypic antibody with binding specificity for said alternative scaffold comprised in said drug molecule. In another preferred embodiment, the drug molecule comprises an alternative scaffold, such as, e.g., an ankyrin repeat domain, and the binding moiety is an anti-idiotypic alternative scaffold, such as, e.g. an ankyrin repeat domain, with binding specificity for said alternative scaffold comprised in said drug molecule.

The term “nucleic acid” or “nucleic acid molecule” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid molecule may either be present in isolated form or be comprised in recombinant nucleic acid molecules or vectors.

The term “biological target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In some embodiments, the biological target is an immune cell, such as a T cell, a B cell, a natural killer (NK) cell, or another type of immune cell. In some other embodiments, the biological target is a tumor cell.

In the context of the present invention, the term “polypeptide” relates to a molecule consisting of a chain of multiple, i.e., two or more, amino acids linked via peptide bonds. Preferably, a polypeptide consists of more than eight amino acids linked via peptide bonds. The term “polypeptide” also includes multiple chains of amino acids, linked together by S-S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (Binz et al., J. Mol. Biol. 332, 489-503, 2003; Binz et al., Nature Biotech. 22(5): 575-582 (2004); WO2002/020565; WO2012/069655), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units. Ankyrin repeat domains may be modularly assembled into larger ankyrin repeat proteins, optionally with half-life extension domains, using standard recombinant DNA technologies (see, e.g., Forrer, P., et al., FEBS letters 539, 2-6, 2003, WO2002/020565, WO2016/156596, WO2018/054971).

The term “designed” as used in designed ankyrin repeat protein and designed ankyrin repeat domain and the like refers to the property that such repeat proteins and repeat domains, respectively, are man-made and do not occur in nature. The designed repeat proteins described herein comprise at least one designed repeat domain. Preferably, the designed repeat domain is a designed ankyrin repeat domain.

The term “target interaction residues” refers to amino acid residues of a binding moiety which contribute to the direct interaction with a drug molecule. For example, if a binding moiety is a designed ankyrin repeat domain, then the term “target interaction residues” refers to amino acid residues of the designed ankyrin repeat domain which contribute to the direct interaction with a drug molecule.

The terms “framework residues” or “framework positions” refer to amino acid residues of a repeat module, which contribute to the folding topology, i.e., which contribute to the fold of said repeat module or which contribute to the interaction with a neighbouring module. Such contribution may be the interaction with other residues in the repeat module, or the influence on the polypeptide backbone conformation as found in a-helices or β-sheets, or the participation in amino acid stretches forming linear polypeptides or loops. Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of designed repeat domains, which are originally derived from the repeat units of naturally occurring repeat proteins. Each repeat module comprised in a repeat domain is derived from one or more repeat units of a family or subfamily of naturally occurring repeat proteins, preferably the family of ankyrin repeat proteins. Furthermore, each repeat module comprised in a repeat domain may comprise a “repeat sequence motif” deduced from homologous repeat modules obtained from repeat domains selected on a target and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art. Designed ankyrin repeat proteins have been described previously; see, e.g., International Patent Publication Nos. WO2002/020565, WO2010/060748, WO2011/135067, WO2012/069654, WO2012/069655, WO2014/001442, WO2014/191574, WO2014/083208, WO2016/156596, and WO2018/054971, all of which are incorporated by reference in their entireties. Typically, an ankyrin repeat module comprises about 31 to 33 amino acid residues that form two alpha helices, separated by loops.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

A binding moiety “specifically binds” or “preferentially binds” (used interchangeably herein) to a drug molecule if it reacts or associates more frequently, more rapidly, with greater duration, with greater affinity and/or with greater avidity with a particular drug molecule than it does with alternative targets (e.g., cells or substances). Binding moieties can be tested for specificity of binding by comparing binding to an appropriate drug molecule to binding to an alternate drug molecule under a given set of conditions. In some embodiments, if the binding molecule binds to the appropriate drug molecule with at least 2-fold, at least 5-fold, at least 10-fold, at least 100-fold, or at least 1000-fold higher affinity than to the alternate drug molecule, then it is considered to be specific. It is also understood by reading this definition that, for example, a binding moiety which specifically or preferentially binds to a first drug molecule may or may not specifically or preferentially bind to a second drug molecule. As such, “specific binding” does not necessarily require (although it can include) exclusive binding. In general, under designated assay conditions, a binding moiety binds preferentially to a particular drug molecule and does not bind in a significant amount to other components present in a test sample.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {⇌}_{{k}_{off}}^{{k}_{on}} [ BT ]$$

kₒₙ is a second-order rate constant of the binding reaction, with the unit M⁻¹ s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function. Formation of the prodrug complex is therefore governed by kₒₙ and k_{off}.

The half-life of the prodrug complex is referred to herein as the “blocking half-life” or “blocking T_{1/2}”, since the prodrug complex (or the binding moiety therein) “blocks” or “inhibits” the mode of action of the drug. Blocking T_{1/2} can be calculated according to the following formula:

$$blocking {T}_{1 / 2} = \frac{ln ( 2 )}{{k}_{off}}$$

where “In(2)” is the natural log (logarithmus naturalis) of the number two, approximately 0.693. It can similarly be said that a binding moiety has a blocking half-life (or blocking T_{1/2}) when complexed with a drug molecule in a composition of the invention. A variety of assay formats may be used to select or characterize a binding moiety that specifically binds a drug molecule of interest. For example, solid-phase ELISA immunoassay, immunoprecipitation, BIAcore™ (GE Healthcare, Piscataway, NJ), fluorescence-activated cell sorting (FACS), Octet™ (ForteBio, Inc., Menlo Park, CA) and Western blot analysis are among many assays that may be used to identify a binding moiety that specifically binds to a target drug molecule. Typically, a specific or selective binding will be at least twice the background signal or noise and more typically more than 10 times the background signal. More particularly, a binding moiety is said to “specifically bind” a target when the equilibrium dissociation constant (K_{D}) value is <1 μM, such as <1 μM, <100 nM, <10 nM, <1 nM, <100 pM, <10 pM, or <1 pM.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). K_{D} value may be measured using OCTET® technology (Octet QKe system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

The term “PBS” means a phosphate buffered water solution containing 137 mM NaCl, 10 mM phosphate and 2.7 mM KCl and having a pH of 7.4.

As used herein, the term “substantially” means at least 95%. Thus, as used herein, the phrase “substantially all of the drug molecules are bound by the binding moiety” and the like means that at least 95% of all of the drug molecules are bound by the binding moiety. In some embodiments, “substantially” means at least 96%, or at least 97%, or at least 98%, or at least 99%. In some embodiments, “substantially” means at least 99.9%.

The term “treat,” as well as words related thereto, does not necessarily imply 100% or complete cure. Rather, there are varying degrees of treatment of which one of ordinary skill in the art recognizes as having a potential benefit or therapeutic effect. In this respect, the methods of treatment and medical uses described herein can provide any amount or any level of treatment. Furthermore, the treatment provided by the method of the present disclosure can include treatment of (i.e., relief from) one or more conditions or symptoms. In exemplary aspects, the invention provides methods of treatment with a drug molecule comprising the administration of a prodrug complex to a patient, wherein the adverse effects, or the risk thereof, experienced by the patient are reduced compared to the adverse effects, or the risk thereof, the patient would experience if the same amount of drug molecule was administered without being in a prodrug complex (i.e., in a complex with a binding moiety of the invention). Thus, the use of a binding moiety of the invention to form a prodrug complex allows methods of treatment with reduced adverse effects, or a reduced risk thereof, and/or methods of treatment with a higher dose of the drug molecule or with administration of the drug molecule over a shorted period of time.

Therapeutic responses in any given disease or condition can be determined by standardized response criteria specific to that disease or condition. The subject undergoing therapy may experience the beneficial effect of an improvement in the symptoms associated with the disease, together with a reduction in adverse effects, or the risk thereof, associated with administration of the therapeutic agent.

As used herein, the term “proliferative disease” refers to diseases characterised by excessive production of cells. Examples of proliferative diseases include, but are not limited to, cancer, atherosclerosis, rheumatoid arthritis, psoriasis, idiopathic pulmonary fibrosis, scleroderma and cirrhosis of the liver. In a preferred embodiment, the proliferative disease is cancer.

### Binding Moieties

The binding moieties described herein are polypeptides or proteins with a variety of different structures, which can bind with high affinity to a drug molecule. Examples of binding moieties for use in the present invention include antibodies, alternative scaffolds, and polypeptides.

Antibodies include any polypeptides or proteins comprising an antigen binding domain that is derived from an antibody or immunoglobulin molecule. The antigen binding domain can be derived, for example, from monoclonal antibodies, polyclonal antibodies, recombinant antibodies, human antibodies, humanized antibodies, and single-domain antibodies, e.g., a heavy chain variable domain (VH), a light chain variable domain (VL) and a variable domain (VHH) from, e.g., human or camelid origin. In some instances, it is beneficial for the antigen binding domain to be derived from the same species in which the binding moiety will ultimately be used in. For example, for use in humans, it may be beneficial for the antigen binding domain of the binding moiety described herein, to comprise a human or a humanized antigen binding domain. Antibodies can be obtained using techniques well known in the art.

In one embodiment, the binding moiety is a camelid nanobody. Camelid nanobodies (also known as camelid single-domain antibodies or VHHs) are derived from the Camelidae family of mammals such the llamas, camels, and alpacas. Unlike other antibodies, camelid antibodies lack a light chain and are composed of two identical heavy chains. Camelid antibodies typically have a relatively low molecular weight in the region of around 15 kDa.

In one embodiment, the binding moiety is a shark antibody domain. Shark antibody domains, like camelid nanobodies, also lack a light chain.

Alternative scaffolds include any polypeptides or proteins comprising a binding domain that is capable of binding an antigen (such as a drug molecule) and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin® proteins), fynomers, knottins, and Kunitz domain peptides. Alternative scaffolds are described, e.g., in Yu et al., Annu Rev Anal Chem (Palo Alto Calif). 2017 Jun. 12; 10(1): 293-320. doi:10.1146/annurevanchem-061516-045205.

Adnectins are originally derived from the tenth extracellular domain of human fibronectin type III protein (10Fn3). The fibronectin type III domain has 7 or 8 beta strands, which are distributed between two beta sheets, which themselves pack against each other to form the core of the protein, and further contain loops (analogous to CDRs), which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, this non-antibody scaffold mimics antigen binding properties that are similar in nature and affinity to those of antibodies. These scaffolds can be used in a loop randomization and shuffling strategy in vitro that is similar to the process of affinity maturation of antibodies in vivo.

Affibody affinity ligands are composed of a three-helix bundle based on the scaffold of one of the IgG-binding domains of Protein A, which is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, but are considerably smaller, having a molecular weight of around 6 kDa, compared to around 150 kDa for antibodies. Despite the size difference, the binding site of affibody molecules has similarity to that of an antibody.

Affilins are synthetic antibody mimetics that are structurally derived from human ubiquitin (historically also from gamma-B crystallin). Affilins consists of two identical domains with mainly beta sheet structure and a total molecular mass of about 20 kDa. They contain several surface-exposed amino acids that are suitable for modification. Affilins resemble antibodies in their affinity and specificity to antigens but not in structure.

Affimers are a type of peptide aptamer, having a structure known as SQT (Stefin A quadruple mutant-Tracy). Aptamers and affimers are short peptides responsible for affinity binding with an inert and rigid protein scaffold for structure constraining in which both N- and C-termini of the binding peptide are embedded in the inert scaffold.

Affitins are variants of the DNA binding protein Sac7d that are engineered to obtain specific binding affinities. Sac7d is originally derived from the hyperthermophile archaea Sulfolobus acidocaldarius and binds with DNA to prevent it from thermal denaturation. Affitins are commercially known as Nanofitins.

Alphabodies are small (approximately 10 kDa) proteins that are engineered to bind to a variety of antigens and are therefore antibody mimetics. The alphabody scaffold is computationally designed based on coiled-coil structures. The standard alphabody scaffold contains three α-helices, composed of four heptad repeats (stretches of 7 residues) each, connected via glycine/serine-rich linkers. The standard heptad sequence is “IAAIQKQ”. Alphabodies' ability to target extracellular and intracellular proteins in combination with their high binding affinities may allow them to bind to targets that cannot be reached with antibodies.

Anticalins are a group of binding proteins with a robust and conservative β-barrel structure found in lipocalins. Lipocalins are a class of extracellular proteins comprising one peptide chain (150-190 amino acids) that is in charge of recognition, storage, and transport of various biological molecules such as signalling molecules.

Armadillo repeat protein-based scaffolds are abundant in eukaryotes and are involved in a broad range of biological processes, especially those related to nuclear transport. Armadillo repeat protein-based scaffolds usually consist of three to five internal repeats and two capping elements. They also have a tandem elongated superhelical structure that enables binding with their corresponding peptide ligands in an extended conformation.

Atrimers are a scaffold derived from a trimeric plasma protein known as tetranectin, belonging to a family of C-type lectins consisting of three identical units. The structure of the C-type lectin domain (CTLD) within the tetranectin has five flexible loops that mediate interaction with targeting molecules.

Avimers are derived from natural A-domain containing proteins such as HER3 and consist of a number of different “A-domain” monomers (2-10) linked via amino acid linkers. Avimers can be created that can bind to the target antigen using the methodology described in, for example, U.S. Patent Application Publication Nos. 2004/0175756; 2005/0053973; 2005/0048512; and 2006/0008844.

In one embodiment, the binding moiety is an ankyrin repeat protein. Designed or engineered ankyrin repeat proteins (such as DARPin® proteins) can function like antibody mimetic proteins, typically exhibiting highly specific and high-affinity target binding. Designed ankyrin repeat proteins comprise one or more designed ankyrin repeat domains. Designed ankyrin repeat domains are derived from natural ankyrin repeat proteins and each designed ankyrin repeat domain typically binds a target protein with high specificity and affinity. Due to their high specificity, stability, potency and affinity and due to their flexibility in formatting to generate mono-, bi- or multi-specific proteins, designed ankyrin repeat proteins are particularly suitable for use as high-affinity binding moieties. Designed ankyrin repeat protein drug candidates also display favourable development properties including rapid, low-cost and high-yield manufacturing and up to several years of shelf-life at 4° C. Designed ankyrin repeat proteins are a preferred embodiment of binding moieties of the invention. DARPin® is a registered trademark owned by Molecular Partners AG.

Fynomers are small globular proteins (approximately 7 kDa) that evolved from amino acids 83-145 of the Src homology domain 3 (SH3) of the human Fyn tyrosine kinase. Fynomers are attractive binding molecules due to their high thermal stability, cysteine-free scaffold, and human origin, which reduce potential immunogenicity.

Knottins, also known as cysteine knot miniproteins, are typically proteins 30 amino acids in length comprising three antiparallel β-sheets and constrained loops laced by a disulfide bond, which creates a cysteine knot. This disulfide bond confers high thermal stability making knottins attractive antibody mimetics.

Kunitz domain peptides or Kunitz domain inhibitors are a class of protease inhibitors with irregular secondary structures containing ˜60 amino acids with three disulfide bonds and three loops that can be mutated without destabilizing the structural framework.

In one embodiment, the binding moiety is a polypeptide or protein comprising an antigen binding domain derived from a T cell receptor (TCR).

### Examples of Binding Moieties

Examples of ankyrin repeat domains for use in the present invention are provided by SEQ ID NOs: 1 to 10 (see the Examples).

The ankyrin repeat domains of SEQ ID NOs: 1 to 10 specifically bind with high affinity to a CD3-specific binding molecule having an amino acid sequence selected from SEQ ID NOs: 12 to 15. For example, the ankyrin repeat domains of SEQ ID NOs: 1 to 10 specifically bind with high affinity to a CD3-specific binding molecule having the amino acid sequence of SEQ ID NO: 13 or the amino acid sequence of SEQ ID NO: 14. In one embodiment, the ankyrin repeat domains of SEQ ID NOs: 1 to 10 specifically bind to a CD3-specific binding molecule having the amino acid sequence of SEQ ID NO: 13. In another embodiment, the ankyrin repeat domains of SEQ ID NOs: 1 to 10 specifically bind to a CD3-specific binding molecule having the amino acid sequence of SEQ ID NO: 14. The CD3-specific binding molecules having an amino acid sequence selected from SEQ ID NOs: 12 to 15 can be genetically fused to another binding molecule with specificity for a tumor-associated antigen (TAA) to form T cell engager (TCE) drug molecules. Binding of an ankyrin repeat domain of any of SEQ ID NOs: 1 to 10 to such a T cell engager drug molecule inhibits binding of the T cell engager drug molecule to CD3 and hence inhibits the biological activity of the T cell engager drug molecule.

Thus, in one embodiment, the binding moiety of the invention is an ankyrin repeat domain comprising an amino acid sequence that has at least about 85% sequence identity with an ankyrin repeat domain selected from the group consisting of SEQ ID NOs: 1 to 10.

In one embodiment, the binding moiety is an ankyrin repeat domain comprising an amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity with an ankyrin repeat domain selected from the group consisting of SEQ ID NOs: 1 to 10.

In one embodiment, the binding moiety is an ankyrin repeat domain, wherein said ankyrin repeat domain is selected from the group consisting of SEQ ID NOs: 1 to 10.

In one embodiment, the binding moiety is a designed ankyrin repeat domain comprising an amino acid sequence selected from the group consisting of (1) SEQ ID NOs: 1 to 10 and (2) sequences that have at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% amino acid sequence identity with any of SEQ ID NOs: 1 to 10.

In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 9 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 8 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 7 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 6 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 5 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 4 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 3 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 2 amino acids in any of SEQ ID NOs: 30 to 51 are substituted by another amino acid. In one embodiment, the binding moiety is a designed ankyrin repeat protein comprising an ankyrin repeat module comprising an amino acid sequence selected from the group consisting of (1) SEQ ID NOs: 30 to 51 and (2) sequences in which up to 1 amino acid in any of SEQ ID NOs: 30 to 51 is substituted by another amino acid.

In all of the binding moieties described herein, the amino acid sequences of the binding moieties may be substituted by one or more amino acids. In some embodiments, up to 15, up to 14, up to 13, up to 12, up to 11, up to 10, up to 9, up to 8, up to 7, up to 6, up to 5, up to 4, up to 3, up to 2, or up to 1 substitution is made in any of the binding moieties described herein.

In some embodiments, up to 15, up to 14, up to 13, up to 12, up to 11, up to 10, up to 9, up to 8, up to 7, up to 6, up to 5, up to 4, up to 3, up to 2, or up to 1 substitution is made in any ankyrin repeat domain relative to any of the sequences of SEQ ID NOs: 1 to 10. In some embodiments, up to 15 substitutions are made relative to any of the sequences of SEQ ID NOs: 1 to 10. In some embodiments, up to 14 substitutions are made relative to any of the sequences of SEQ ID NOs: 1 to 10. In some embodiments, up to 13 substitutions are made relative to any of the sequences of SEQ ID NOs: 1 to 10. In some embodiments, up to 12 substitutions are made relative to any of the sequences of SEQ ID NOs: 1 to 10. In some embodiments, up to 11 substitutions are made relative to any of the sequences of SEQ ID NOs: 1 to 10. In some embodiments, up to 10 substitutions are made relative to any of the sequences of SEQ ID NOs: 1 to 10. In some embodiments, up to 9 substitutions are made relative to any of the sequences of SEQ ID NOs: 1 to 10. In some embodiments, up to 8 substitutions are made relative to any of the sequences of SEQ ID NOs: 1 to 10. In some embodiments, up to 7 substitutions are made relative to any of the sequences of SEQ ID NOs: 1 to 10. In some embodiments, up to 6 substitutions are made relative to any of the sequences of SEQ ID NOs: 1 to 10. In some embodiments, up to 5 substitutions are made relative to any of the sequences of SEQ ID NOs: 1 to 10. In some embodiments, up to 4 substitutions are made relative to any of the sequences of SEQ ID NOs: 1 to 10. In some embodiments, up to 3 substitutions are made relative to any of the sequences of SEQ ID NOs: 1 to 10. In some embodiments, up to 2 substitutions are made relative to any of the sequences of SEQ ID NOs: 1 to 10. In some embodiments, up to 1 substitution is made relative to any of the sequences of SEQ ID NOs: 1 to 10.

In some embodiments, the amino acid substitution(s) are all made in framework positions. In some embodiments, the amino acid substitution(s) are all made in non-randomized positions. The location of randomized positions in a designed ankyrin repeat domain is disclosed, e.g., in Binz et al., Nature Biotech. 22(5): 575-582 (2004).

In some embodiments, the amino acid substitution(s) do not change the K_{D} value by more than about 1000-fold, more than about 100-fold, or more than about 10-fold, compared to the K_{D} value of the unsubstituted binding moieties. For example, in some embodiments, the amino acid substitution(s) do not change the K_{D} value by more than about 1000-fold, more than about 300-fold, more than about 100-fold, more than about 50-fold, more than about 25-fold, more than about 10-fold, or more than about 5-fold, compared to the K_{D} value of the binding of a binding moiety comprising any of the sequences of SEQ ID NOs: 1 to 10 to a drug molecule comprising any of the sequences of SEQ ID NOs: 12 to 15.

In certain embodiments, the amino acid substitution in the binding moiety is a conservative substitution according to Table 1 below.

TABLE 1
Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; Norleucine |

When the binding moiety is an ankyrin repeat domain, in some embodiments, the substitution may be made outside the structural core residues of the ankyrin repeat domain, e.g., in the beta loops that connect the alpha-helices. In other embodiments, the substitution may be made within the structural core residues of the ankyrin repeat domain. For example, the ankyrin domain may comprise the consensus sequence: xDxxGxTPLHLAxxxGxxxIVxVLLxxGADVNA (SEQ ID NO: 52), wherein “x” denotes any amino acid (preferably not cysteine, glycine, or proline); or xDxxGxTPLHLAAxxGHLEIVEVLLKzGADVNA (SEQ ID NO: 53), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made outside the residues designated as “x”.

In addition, the second last position of any ankyrin repeat domain of a binding moiety can be “A” or “L”, and/or the last position can be “A” or “N”. Accordingly, in some embodiments, an ankyrin repeat domain of a binding moiety comprises an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to any one of SEQ ID NOs: 1 to 10, and wherein optionally A at the second last position is substituted with L and/or A at the last position is substituted with N. In an exemplary embodiment, an ankyrin repeat domain of a binding moiety comprises an amino acid sequence that is at least about 90% identical to any one of SEQ ID NOs: 1 to 10, and wherein optionally A at the second last position is substituted with L and/or A at the last position is substituted with N. Furthermore, the sequence of any ankyrin repeat domain of a binding moiety may optionally comprise at its N-terminus, a G, an S, or a GS (see below).

In addition, each ankyrin repeat domain of a binding moiety may optionally comprise a “G,” an “S,” or a “GS” sequence at its N-terminus. Accordingly, in some embodiments, an ankyrin repeat domain of a binding moiety comprises an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to any one of SEQ ID NOs: 1 to 10, and further optionally comprises a G, an S, or a GS at its N-terminus. In an exemplary embodiment, an ankyrin repeat domain of a binding moiety comprises an amino acid sequence that is at least about 90% identical to any one of SEQ ID NOs: 1 to 10, and wherein said ankyrin repeat domain further optionally comprises a G, an S, or a GS at its N-terminus. Furthermore, the sequence of any ankyrin repeat domain of a binding moiety may optionally have A at the second last position substituted with L and/or A at the last position substituted with N (see above).

### N-Terminal and C-Terminal Capping Sequences

When the binding moieties described herein comprise ankyrin repeat domains, the ankyrin repeat domains may comprise N-terminal or C-terminal capping sequences. Capping sequences refer to additional polypeptide sequences fused to the N- or C-terminal end of ankyrin repeat sequence motif(s) or module(s), wherein said capping sequences form tight tertiary interactions (i.e., tertiary structure interactions) with neighbouring ankyrin repeat sequence motif(s) or module(s) of the ankyrin repeat domains, thereby providing a cap that shields the hydrophobic core of the ankyrin repeat domain at the side from exposure to solvent.

The N- and/or C-terminal capping sequences may be derived from, a capping unit or other structural unit found in a naturally occurring repeat protein adjacent to a repeat unit. Examples of capping sequences are described in International Patent Publication Nos. WO2002/020565 and WO2012/069655, in U.S. Patent Publication No. US 2013/0296221, and by Interlandi et al., J Mol Biol. 2008 Jan 18;375(3):837-54.

Examples of N-terminal ankyrin capping modules (i.e., N-terminal capping repeats) are SEQ ID NOs: 21 to 23 and examples of C-terminal capping modules (i.e., C-terminal capping repeats) includes SEQ ID NO: 28.

### Drug Molecules

Within the context of the present invention, drug molecules are therapeutic agents that comprise a polypeptide or a protein, wherein said polypeptide or protein contains a site that is capable of being bound by a binding moiety. There is no particular limitation on the drug molecules for use in the present invention, provided that these can be bound by a binding moiety. This means that, for example, the drug molecule may belong to the same “class” as the binding moiety or to a different “class” as the binding moiety, such that, for example, both the drug molecule and the binding moiety may be antibodies, or both the drug molecule and the binding moiety may be alternative scaffolds (e.g. ankyrin repeat proteins), or the drug molecule may be an antibody and the binding moiety may be an alternative scaffold (e.g. an ankyrin repeat protein), or the drug molecule may be an alternative scaffold (e.g. an ankyrin repeat protein) and the binding moiety may be an antibody. This further means, for example, that the drug molecule itself may comprise different structural moieties, for example, combining an antibody moiety and an alternative scaffold moiety, or combining moieties of different alternative scaffold structures. In case both the drug molecule and the binding moiety are antibodies, it would be clear to the skilled person that the antibodies would be different from each other, including with respect to binding specificity. Similarly, in case both the drug molecule and the binding moiety are alternative scaffolds, it would be clear to the skilled person that the alternative scaffolds would be different from each other, including with respect to binding specificity.

Furthermore, a drug molecule for use in the present invention may contain a half-life extending moiety. A half-life extending moiety extends the serum half-life in vivo of a drug molecule, compared to the same molecule without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), human serum albumin (HSA) binding domain, or polyethylene glycol (PEG). In some instances, the half-life extending moiety may comprise an ankyrin repeat domain with binding specificity for HSA. In other instances, the half-life extending moiety may comprise an immunoglobulin domain, such as an Fc domain, e.g., the Fc domain of human IgG₁, or a variant or derivative thereof.

In some embodiments, drug molecules for use in the present invention comprise alternative scaffolds, wherein the alternative scaffolds are selected from adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin® proteins), fynomers, knottins, and Kunitz domain peptides. Alternative scaffolds are described, e.g., in Yu et al., Annu Rev Anal Chem (Palo Alto Calif). 2017 Jun. 12; 10(1): 293-320. doi:10.1146/annurevanchem-061516-045205.

Drug molecules for use in the present invention also include, but are not limited to, different categories of drugs that are currently approved for clinical use, such as:
(1) immune-checkpoint inhibitors (ICIs);
(2) bispecific antibodies; and
(3) genetically modified immune cells, such as T cells, in particular, chimeric antigen receptor (CAR)-expressing immune cells, such as CAR-T cells.

Drug molecules for use in the present invention also include, but are not limited to, drug molecules that up- or down-regulate the activity of immune checkpoints, herein called “immune checkpoint regulators”. Immune checkpoints are molecules in the immune system that either turn up (co-stimulatory molecules) or turn down (inhibitory molecules) immune signals. In cancer patients, tumors can use these immune checkpoints to protect themselves from immune system attacks, particularly by T cells. Drug molecules used in immune checkpoint therapy can block inhibitory immune checkpoint molecules or activate stimulatory immune checkpoint molecules, thereby restoring immune system function. In recent years, immune checkpoint drugs have become important novel cancer treatment options. Immune checkpoint molecules include, but are not limited to, CD27, CD137, CD137L, 2B4, TIGIT, CD155, ICOS, HVEM, CD40L, LIGHT, TIM-1, OX40, OX40L, DNAM-1, PD-L1, PD1, PD-L2, CTLA-4, CD8, CD40, CEACAMI, CD48, CD70, A2AR, CD39, CD73, B7-H3, B7-H4, BTLA, C5AR1, CCR8, CD226, CD28, CD33, CD38, CD3e, CD47, CD94, ETAR, NKG2A, SIRPα, TLR8, TNFRSF18, IDO1, IDO2, TDO, KIR, LAG-3, TIM-3, TIM-4 and VISTA. In one embodiment, the drug molecule is an immune-checkpoint regulator.

In one embodiment, the drug molecule for use in the present invention comprises an antibody. In another embodiment, the drug molecule comprises a bispecific antibody. In another embodiment, the drug molecule comprises a multispecific antibody. In another embodiment, the drug molecule comprises an antibody that is a T-cell engager drug molecule (TCE).

Bispecific antibodies include TCEs. An example of bispecific antibodies that are TCEs are the molecules known as BiTE™ molecules. These are anti-cancer drugs consisting of two single-chain variable fragments (scFvs) on a single peptide chain. Such TCEs bind to the CD3 (cluster of differentiation 3) molecule on the surface of T cells through one of the scFvs, while the other scFv binds to a tumor-associated antigen (TAA) on the surface of tumor cells. By binding to CD3 and linking a T cell to a tumor cell, the T cell is “activated” and can exert cytotoxic activity on the tumor cell. One example of a bispecific antibody that is a TCE is blinatumomab, which binds to CD3 on the surface of T cells and to CD19 on the surface of B cells. Blinatumomab is approved for use in the treatment of acute lymphoblastic leukaemia.

In one embodiment, the drug molecule for use in the present invention comprises an alternative scaffold. In another embodiment, the drug molecule comprises a bispecific alternative scaffold. In another embodiment, the drug molecule comprises a multispecific alternative scaffold. In another embodiment, the drug molecule comprises an alternative scaffold molecule that is a T-cell engager drug molecule (TCE). In a preferred embodiment, said alternative scaffold is an ankyrin repeat domain. In a further preferred embodiment, the drug molecule for use in the present invention comprises an alternative scaffold, wherein said alternative scaffold is an ankyrin repeat domain having binding specificity for CD3, and wherein said ankyrin repeat domain comprises an amino acid sequence selected from SEQ ID NOs: 12 to 15. In a further preferred embodiment, the drug molecule comprises an ankyrin repeat domain having binding specificity for CD3, wherein said ankyrin repeat domain comprises an amino acid sequence selected from SEQ ID NOs: 12 to 15. In one embodiment, the drug molecule comprises an ankyrin repeat domain having binding specificity for CD3, wherein said ankyrin repeat domain comprises an amino acid sequence selected from SEQ ID NOs: 13 to 14. In one embodiment, the drug molecule comprises an ankyrin repeat domain having binding specificity for CD3, wherein said ankyrin repeat domain comprises SEQ ID NO: 13. In one embodiment, the drug molecule comprises an ankyrin repeat domain having binding specificity for CD3, wherein said ankyrin repeat domain comprises SEQ ID NO: 14.

Bispecific or multispecific alternative scaffold molecules include TCEs. In one embodiment, the drug molecule is a bispecific or multispecific alternative scaffold molecule, wherein said alternative scaffold molecule is a TCE comprising (i) a CD3-specific binding domain that is an ankyrin repeat domain, and (ii) a TAA-specific binding domain that is an ankyrin repeat domain. Similar to TCEs known as BiTE™ molecules, such TCEs comprising alternative scaffolds are anti-cancer drugs that bind to the CD3 molecule on the surface of T cells through one of the binding domains, while the other binding domain binds to a tumor-associated antigen (TAA) on the surface of tumor cells. By binding to CD3 and linking a T cell to a tumor cell, the T cell is “activated” and can exert cytotoxic activity on the tumor cell.

When the drug molecule is a TCE, the preferred binding site for the binding moiety of the invention is the CD3-specific binding domain of the TCE. By binding to the CD3-specific binding domain of the TCE, the binding moiety blocks the mode of action of the TCE by preventing the TCE from binding to T cells. In one embodiment, the binding of the binding moiety to the CD3-specific binding domain of the TCE is anti-idiotypic.

Numerous bispecific antibodies that are TCEs have been described, including those listed below, with their respective binding targets provided in brackets. For example, as described above, blinatumomab (CD19xCD3; Amgen) binds to the CD3 antigen on a T cell, and to a CD19 antigen on a tumor cell that arose from the B cell lineage. Other bispecifics that are TCEs include, but are not limited to, AMG330 (CD33xCD3; Amgen); flotetuzumab (CD123xCD3; Macrogenics); MCLA117 (Clec12AXCD3; Merus); AMG160 (HLE PSMAxCD3, Amgen); AMG427 (HLE FLT3xCD3, Amgen); AMG562 (HLE CD19xCD3, Amgen); AMG596 (HLE EGFRvIIIxCD3, Amgen); AMG673 (HLE CD33xCD3, Amgen); AMG701 (HLE BCMAxCD3, Amgen); AMG757 (HLE DLL3xCD3, Amgen); AMG910 (HLE Claudin18.2xCD3, Amgen); odronextamab (CD20xCD3, Regeneron); mosunetuzumab (CD20xCD3, Roche); glofitamab (CD20xCD3, Roche); and epcoritamab (CD20xCD3, Genmab). Any of such TCEs can be used as a drug molecule in a composition of the invention.

In one embodiment, the drug molecule for use in the present invention comprises an antibody and an alternative scaffold. In another embodiment, the drug molecule comprises two different alternative scaffolds. In another embodiment, the drug molecule comprises a T cell receptor (TCR)-derived antigen-recognition domain.

In one embodiment, the drug molecule for use in the present invention comprises genetically modified immune cells. In a preferred embodiment, said genetically modified immune cells express a chimeric antigen receptor (CAR). In one embodiment, said genetically modified immune cells are genetically modified T cells, such as CAR-expressing T cells (CAR-T cells). In another embodiment, said genetically modified immune cells are genetically modified natural killer (NK) cells, such as CAR-expressing NK cells (CAR-NK cells).

### Binding Affinity

The binding moiety and drug molecules described herein bind in a non-covalent fashion to form a prodrug complex. Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {⇌}_{{k}_{off}}^{{k}_{on}} [ BT ]$$

In certain embodiments, the binding affinity of the binding moiety to the drug molecule is described in terms of K_{D}. In exemplary embodiments, the K_{D} is about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻¹¹ M to about 10⁻¹⁵ M, from about 10⁻¹² M to about 10⁻¹⁵ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻¹¹ M to about 10⁻¹⁴ M, from about 10⁻¹² M to about 10⁻¹⁴ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻¹¹ M to about 10⁻¹³ M, or from about 10⁻¹² M to about 10⁻¹³ M.

In exemplary embodiments, the binding moiety binds to the drug molecule with a K_{D} value of, or less than: about 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In one exemplary embodiment, the binding moiety binds to the drug molecule with a K_{D} value of less than or equal to about 1 nM. In another exemplary embodiment, the binding moiety binds to the drug molecule with a K_{D} value of less than or equal to about 100 pM. In another exemplary embodiment, the binding moiety binds to the drug molecule with a K_{D} value of less than or equal to about 10 pM. In yet another exemplary embodiment, the binding moiety binds to the drug molecule with a K_{D} value of less than or equal to about 1 pM.

In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with SEQ ID Nos: 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with SEQ ID NOs 13 or 14, wherein said binding moiety binds to said drug molecule with a KD value in the range of about 1 to about 100 pM.

In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with any of SEQ ID NOs 12 to 15, wherein said binding moiety binds to said drug molecule with a K_{D} value of less than about 100 nm, such as less than about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM, about 10 pM, about 5 pM, about 2 pM, about 1 pM, about 500 fM, about 250 fM, about 100 fM, about 50 fM, about 25 fM, about 10 fM, about 5 fM, about 2 fM, or about 1 fM. In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with SEQ ID NOs 13 or 14, wherein said binding moiety binds to said drug molecule with a K_{D} value in the range of about 1 to about 100 pM.

In exemplary embodiments, the binding moiety binds to the drug molecule with a K_{off} value of between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹ or between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹.

In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with any of SEQ ID NOs 12 to 15, wherein said binding moiety binds to said drug molecule with a koff value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with SEQ ID NOs 13 or 14, wherein said binding moiety binds to said drug molecule with a koff value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with any of SEQ ID NOs 12 to 15, wherein said binding moiety binds to said drug molecule with a k_{off} value between about 1×10⁻⁸ s⁻¹ and about 1×10⁻⁴ s⁻¹, such as between about 1×10⁻⁸ s⁻¹ and about 1×10⁻⁷ s⁻¹, between about 1×10⁻⁸ s⁻¹ and about 1×10⁻⁶ s⁻¹, between about 1×10⁻⁸ s⁻¹ and about 1×10⁻⁵ s⁻¹, between about 1×10⁻⁷ s⁻¹ and about 1×10⁻⁴ s⁻¹, between about 1×10⁻⁷ s⁻¹ and about 1×10⁻⁵ s⁻¹, between about 1×10⁻⁷ s⁻¹ and about 1×10⁻⁶ s⁻¹, between about 1×10⁻⁶ s⁻¹ and about 1×10⁻⁴ s⁻¹, or between about 1×10⁻⁶ s⁻¹ and about 1×10⁻⁵ s⁻¹. In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with SEQ ID NOs 13 or 14, wherein said binding moiety binds to said drug molecule with a k_{off} value between about 1×10⁻⁷ s⁻¹ and about 1×10⁻⁴ s⁻¹.

When the binding moiety and drug molecule are bound together, the drug molecule is unable to exert a biological activity, such as, for example, binding to a biological target molecule. Thus, the activity of the drug is “blocked” while the binding moiety is bound to it. The blocking half-life (referred to as “blocking T_{1/2}” in the following) is the time taken for half of the prodrug complex to disassociate. Thus, T_{1/2} is the “half-life” of the prodrug complex. The blocking T_{1/2} can be calculated according to the following formula:

$$blocking {T}_{1 / 2} = \frac{ln ( 2 )}{{k}_{off}}$$

To achieve slow-release of the drug into the subject, the blocking half-life should be at least about 2 hours, at least about 5 hours, at least about 10 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours. The blocking half-life can fall between any of the above values. For example, the blocking half-life may be in the range of about 10 to about 250 hours, about 20 to about 250 hours, about 40 to about 200 hours, or about 50 to about 100 hours.

In some embodiments, the composition of the invention comprises, consists essentially of, or consists of a binding moiety comprising an ankyrin repeat domain having at least about 85% amino acid sequence identity with SEQ ID NO: 1, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 2, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 3, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 4, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 5, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 6, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 7, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 8, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 9, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

In some embodiments, the composition of the invention comprises, consists essentially of, or consists of a binding moiety comprising an ankyrin repeat domain having at least about 85% amino acid sequence identity with SEQ ID NO: 10, and a drug molecule comprising an ankyrin repeat domain having at least about 85% amino acid sequence identity with any of SEQ ID NOs: 12 to 15, wherein said prodrug complex exhibits a blocking half-life of at least about 2 hours, at least about 3 hours, at least about 4 hours, at least about 5 hours, at least about 6 hours, at least about 7 hours, at least about 8 hours, at least about 9 hours, at least about 10 hours, at least about 15 hours, at least about 20 hours, at least about 25 hours, at least about 30 hours, at least about 35 hours, at least about 40 hours, at least about 45 hours, at least about 50 hours, at least about 60 hours, at least about 70 hours, at least about 80 hours, at least about 90 hours, at least about 100 hours, at least about 150 hours, or at least about 200 hours.

There is no particular upper limit on the blocking half-life. However, for certain disease indications and associated drug molecules the skilled person would understand that, if the blocking half-life of a prodrug complex is too long, the drug may be released into the body too slowly to have the desired therapeutic benefits. Consequently, the blocking half-life should be such that the desired therapeutic benefits of a given drug molecule are still observable.

### Compositions

In one embodiment, the present invention relates to a composition comprising a binding moiety as defined herein; and a drug molecule as defined herein; wherein said binding moiety reversibly binds to the drug molecule; and wherein said binding moiety, when bound, inhibits a biological activity of said drug molecule. In one embodiment, said biological activity is binding of said drug molecule to a biological target. In one embodiment, the binding affinity of said binding moiety to said drug molecule allows release of the drug molecule as a function of time upon administration in vivo. As used herein, the term “composition” is used interchangeably with “prodrug complex”.

Any protein binding moiety listed above may be combined with any drug molecule listed above to form a composition (i.e., a prodrug complex), provided that the binding moiety has the desired binding affinity and specificity for the drug molecule.

The compositions may comprise any of the binding moiety and drug molecule combinations described above, in particular any of the specifically disclosed combinations with a binding coefficient (such as K_{D} and k_{off}) or a blocking half-life explicitly disclosed.

As described above, designed ankyrin repeat domains are preferred binding moieties for use in the present invention. Thus, in one embodiment, the compositions comprise a binding moiety that comprises a designed ankyrin repeat domain. In another embodiment, the compositions comprise a binding moiety that comprises a designed ankyrin repeat domain and a drug molecule that comprises a designed ankyrin repeat domain. In another embodiment, the compositions comprise a binding moiety that comprises a designed ankyrin repeat domain and a drug molecule that comprises two, three, four, five or more designed ankyrin repeat domains. In another embodiment, the compositions comprise a binding moiety that comprises a designed ankyrin repeat domain and a drug molecule that comprises an alternative scaffold. In another embodiment, the compositions comprise a binding moiety that comprises a designed ankyrin repeat domain and a drug molecule that comprises an antibody. In another embodiment, the compositions comprise a binding moiety that comprises a designed ankyrin repeat domain and a drug molecule that comprises an antigen binding domain derived from a T cell receptor (TCR). In one embodiment, the compositions comprise a binding moiety that comprises a designed ankyrin repeat domain and a drug molecule that is a T cell engager drug molecule. In one embodiment, the compositions comprise a binding moiety that comprises a designed ankyrin repeat domain and a drug molecule that is an immune-checkpoint regulator drug molecule. In one embodiment, the compositions comprise a binding moiety that comprises a designed ankyrin repeat protein and a drug molecule that is a bispecific antibody drug molecule. In one embodiment, the compositions comprise a binding moiety that comprises a designed ankyrin repeat domain and a drug molecule that is a CAR-expressing immune cell, such as a CAR-T cell or a CAR-NK cell.

In one embodiment, the composition comprises (i) a binding moiety comprising an ankyrin repeat domain having an amino acid sequence selected from the group consisting of (1) SEQ ID NO: 1 and (2) sequences that have at least about 85% amino acid sequence identity with SEQ ID NO: 1; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 1, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 1, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 2 and (2) sequences that have at least about 85% amino acid sequence identity with SEQ ID NO: 2; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 2, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 2, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 3 and (2) sequences that have at least about 85% amino acid sequence identity with SEQ ID NO: 3; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 3 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 3 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 3 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 3, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 3, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 4 and (2) sequences that have at least about 85% amino acid sequence identity with SEQ ID NO: 4; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 4, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 4, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 5 and (2) sequences that have at least about 85% amino acid sequence identity with SEQ ID NO: 5; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 5, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 5, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 6 and (2) sequences that have at least about 85% amino acid sequence identity with SEQ ID NO: 6; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 6, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 6, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 7 and (2) sequences that have at least about 85% amino acid sequence identity with SEQ ID NO: 7; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 7, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 7, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 8 and (2) sequences that have at least about 85% amino acid sequence identity with SEQ ID NO: 8; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 8, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 8, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 9 and (2) sequences that have at least about 85% amino acid sequence identity with SEQ ID NO: 9; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 9 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 9 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 9 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 9, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 9, and (2) a drug molecule comprising an ankyrin repeat domain comprising any of SEQ ID NOs: 13 and 14.

In one embodiment, the composition comprises (i) a binding moiety comprising an ankyrin repeat domain having an amino acid sequence selected from the group consisting of (1) SEQ ID NO: 10 and (2) sequences that have at least about 85% amino acid sequence identity with SEQ ID NO: 10; and (ii) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 and 14.

In one embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising any of SEQ ID NOs: 12 to 15. In another embodiment, the composition comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising any of SEQ ID NOs: 13 and 14.

In another embodiment, the composition comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising an ankyrin repeat domain having an amino acid sequence selected from the group consisting of (a) SEQ ID NOs: 12 to 15 and (2) sequences that have at least about 85% amino acid sequence identity with SEQ ID NOs: 12 to 15.

In another embodiment, the composition comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising any of SEQ ID NOs: 12 to 15. In another embodiment, the composition comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising any of SEQ ID NOs: 13 to 14.

In another embodiment, the composition comprises (1) a binding moiety comprising an ankyrin repeat domain having SEQ ID NO: 10, and (2) a drug molecule comprising an ankyrin repeat domain comprising any of SEQ ID NOs: 12 to 15. In another embodiment, the composition comprises (1) a binding moiety comprising an ankyrin repeat domain having SEQ ID NO: 10, and (2) a drug molecule comprising an ankyrin repeat domain comprising any of SEQ ID NOs: 13 and 14.

In one embodiment, said drug molecule comprised in any of said compositions described above is a bi- or multi-specific drug molecule. In another embodiment, said drug molecule comprised in any of said compositions described above is a T cell engager drug molecule.

As described above, antibodies and alternative scaffolds are also binding moieties for use in the present invention. Thus, in one embodiment, the compositions comprise a binding moiety that comprises an antibody or an alternative scaffold. In another embodiment, the compositions comprise (1) a binding moiety that comprises an antibody or an alternative scaffold, and (2) a drug molecule that comprises a designed ankyrin repeat domain. In another embodiment, the compositions comprise (1) a binding moiety that comprises an antibody or an alternative scaffold, (2) and a drug molecule that comprises an antibody. In one embodiment, the compositions comprise (1) a binding moiety that comprises an antibody or an alternative scaffold, and (2) a drug molecule that is a T cell engager drug molecule. In one embodiment, the compositions comprise (1) a binding moiety that comprises an antibody or an alternative scaffold, and (2) a drug molecule that is an immune-checkpoint inhibitor drug molecule. In one embodiment, the compositions comprise (1) a binding moiety that comprises an antibody or an alternative scaffold, and (2) a drug molecule that is a bispecific antibody drug molecule. In one embodiment, the compositions comprise (1) a binding moiety that comprises an antibody or an alternative scaffold, and (2) a drug molecule that is an immune cell-activating drug molecule. In one embodiment, the compositions comprise (1) a binding moiety that comprises an antibody or an alternative scaffold, and (2) a drug molecule that is a genetically modified immune cell, such as a CAR-expressing immune cell, such as a CAR-T cell or a CAR-NK cell.

Since the compositions of the present invention require that substantially all of the drug molecules are bound to the binding moiety, it is preferred that the pharmaceutical compositions are given sufficient time for such binding to occur and for an equilibrium to be reached. The time required to form an equilibrium will depend on the binding constants (kₒₙ and k_{off}).

Thus, in one embodiment, the present invention relates to a method of making a controlled release formulation comprising the steps of:
(i) providing a binding moiety as defined herein;
(ii) providing a drug molecule as defined herein; and
(iii) allowing said binding moiety and active drug molecule to reach an equilibrium such that substantially all of the drug molecule is bound to the binding moiety.

In one embodiment, the drug molecule and binding moiety allowed to equilibrate for at least about 1 hour, such as at least about 2 hours, at least about 6 hours, at least about 12 hours, at least about 24 hours, at least about 48 hours, or at least about 72 hours.

The concentration of binding moiety in the composition should be relatively high to ensure that the kₒₙ equilibrium constant is considerably higher than the k_{off} equilibrium constant, meaning that substantially all of the drug molecules are bound by the binding moiety and that the mode of action of the drug molecule is substantially inhibited. In one embodiment, the binding moiety and the drug molecule are provided in a molar ratio of 1:1. In some embodiments, the binding moiety should be present in a molar excess, as compared with the amount of drug molecule. Thus, in these embodiments, the binding moiety is provided in a molar ratio to the drug molecule of at least 1.2:1, at least 1.5:1, at least 2:1, at least 5:1, at least 10:1, at least 20:1, at least 50:1, at least 100:1, at least 200:1, at least 400:1, or at least 1000:1.

The invention further relates to pharmaceutical compositions comprising the composition described herein and a pharmaceutically acceptable carrier or excipient. Uses and methods of treatment using said pharmaceutical compositions are also described herein. The methods and uses encompassed by the present invention are described in more detail below. It is noted that the pharmaceutical compositions, methods and uses treat the disease indications that are treated by the drug molecules used to make the pharmaceutical composition.

The primary function of the binding moieties is to bind to the drug molecules to effect slow release of the drug molecule. Thus, in some embodiments, the binding moieties do not substantially alter the biological effect of the drug molecule in vivo, other than to release the drug molecule into the body at a slower rate than direct administration without prior complexation with a protein binding moiety. The binding moieties may, however, comprise additional functional moieties. Such additional functional moieties may provide specific advantages such as biological activity or may be useful in tagging/labelling of the binding moieties for e.g. detection purposes.

The pharmaceutical compositions described herein may be prepared using methods known in the art.

The pharmaceutical compositions comprise a pharmaceutically acceptable carrier or excipient. Standard pharmaceutical carriers include a phosphate buffered saline solution, water, emulsions such as an oil/water or water/oil emulsion, and various types of wetting agents.

The pharmaceutical compositions can comprise any other pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, colouring agents, desiccants, detergents, diluents, disinfectants, disintegrants, dispersing agents, dissolution enhancing agents, dyes, emollients, emulsifying agents, emulsion stabilizers, fillers, film forming agents, flavour enhancers, flavouring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The pharmaceutical compositions can be formulated to achieve a physiologically compatible pH. In some embodiments, the pH of the pharmaceutical composition can be, for example, between about 4 or about 5 and about 8.0, or between about 4.5 and about 7.5, or between about 5.0 and about 7.5. In exemplary embodiments, the pH of the pharmaceutical composition is between about 5.5 and about 7.5.

In one embodiment, the present invention relates to a method of immune cell activation, such as T cell activation or NK cell activation, in a subject in need thereof, the method comprising the step of administering to said subject the pharmaceutical composition as described herein.

In another embodiment, the present invention relates to a method of controlling release of an active drug molecule in vivo comprising administering the pharmaceutical composition as described herein to a subject in need thereof.

In another embodiment, the present invention relates to a method of treating a subject, the method comprising the step of administering an effective amount of a pharmaceutical composition as defined herein, to a subject in need thereof. In some embodiments, the method is a method of treating a proliferative disease. In some embodiments, the method is a method of treating cancer.

In another embodiment, the present invention relates to a composition as defined herein for use in therapy. In another embodiment, the present invention relates to a pharmaceutical composition as defined herein for use in therapy. Preferably, the pharmaceutical composition as defined herein is provided for use in treating a proliferative disease. In more preferred embodiments, the proliferative disease is cancer.

The pharmaceutical compositions of the invention are typically administered to subjects that have been identified as having a high risk for specific side effects typically associated with the drug molecule and/or as requiring a large dose of the drug molecule such that problematic side effects have an increased likelihood. In some embodiments, the subject is a mammal. In preferred embodiments, the subject is a human.

In some embodiments, a single administration of the pharmaceutical composition may be sufficient. In other embodiments, repeated administration may be necessary. Various factors will impact on the number and frequency of administrations, such as the age and general health of the subject, as well as the nature and typical dosage regime of the drug molecule.

The pharmaceutical compositions described herein can be administered to the subject via any suitable route of administration, such as parenteral, nasal, oral, pulmonary, topical, vaginal, or rectal administration. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. For additional details, see Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

The pharmaceutical compositions described herein may be used in combination with another therapeutic agent. Each therapeutic agent may be administered simultaneously (e.g., in the same medicament or at the same time), concurrently (i.e., in separate medicaments administered one right after the other in any order) or sequentially in any order. Sequential administration may be useful when the therapeutic agents in the combination therapy are in different dosage forms (e.g., one agent is a tablet or capsule and another agent is a sterile liquid) and/or are administered on different dosing schedules, e.g., an analgesic that is administered at least daily and a biotherapeutic that is administered less frequently, such as once weekly or once every two weeks.

Additional therapeutic agents include, but are not limited to, analgesics, steroids, anti-cancer agents, antibiotics, penicillin, anti-hypertensive agents, anti-diabetic agents, anti-convulsants, anti-emetics, anti-depressants, check point inhibitors (e.g. anti-PD-1, PD-L1, Tim-3, LAG-3 etc.), immune agonists (e.g. 41-BB, CD28, CD40 etc.), cytokines (IL-15, IL-12, etc or anti-TGFb, anti-IL-10 etc.), immune stimulatory agents, T-cell stimulatory agents, immune-oncology modalities (e.g. CAR-T and cell therapy, antibody drug conjugates (ADCs), oncolytic viruses etc.), chemotherapy, radioimmunoconjugates, and the like.

### Nucleic Acids & Methods

The present invention further relates to a nucleic acid encoding the binding moiety as described herein. In one embodiment, the nucleic acid encodes a binding moiety comprising a designed ankyrin repeat domain as defined herein. Examples of such nucleic acids are provided by SEQ ID NOs: 16 to 19. The present invention further relates to a host cell comprising said nucleic acid.

The present invention further relates to a method of making the binding moiety as defined herein, comprising culturing the host cell defined herein under conditions wherein said recombinant binding protein is expressed. In some embodiments, said host cell is a eukaryotic host cell. In other embodiments, said host cell is a prokaryotic host cell. In one embodiment, the method of making the binding moiety comprises culturing the host cell under conditions wherein said recombinant binding protein is expressed, wherein said binding moiety comprises a designed ankyrin repeat domain and wherein said host cell is prokaryotic host cell, such as, for example, E. coll. In another embodiment, the method of making the binding moiety comprises culturing the host cell under conditions wherein said recombinant binding protein is expressed, wherein said binding moiety comprises an antibody and wherein said host cell is a eukaryotic host cell, such as, for example, a CHO cell.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g., E. coli XL1-blue (Stratagene, USA) or BL21 (Novagen, USA). NLC chips for SPR measurements were from BioRad (BioRad, USA). HTRF reagents were from Cisbio (Cisbio, France). Pan-T cell isolation kit was from Miltenyi Biotec (Germany). Polyester inlets for the “complex” Incucyte in 24-well format were from Corning (USA), and neutravidin beads were from Thermo Fischer (USA). Nuclight Red Lentivirus was from Sartorius (Germany). Cytotoxicity detection (by LDH release) kit was from Roche.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 21, 22 or 23) or a randomized N-terminal capping module according to SEQ ID NO: 24, one or more randomized repeat modules according to the sequence motif of SEQ ID NO: 25, 26 or 27, and a fixed C-terminal capping module (e.g. the C-terminal capping module of SEQ ID NO: 28) ora randomized C-terminal capping module according to SEQ ID NO: 29. Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g., 1 to 20 amino acids) inserted in one or more beta-turns of an ankyrin repeat domain.

An N-terminal capping module of an ankyrin repeat protein library preferably possesses the RILLAA, RILLKA or RELLKA motif and any such C-terminal capping module of an ankyrin repeat protein library preferably possesses the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

### Example 1: Selection of Ankyrin Repeat Domains with Binding Specificity for CD3-Specific Binding Domains

### Summary

Using ribosome display (Hanes, J. and Plückthun, A., PNAS 94, 4937-42, 1997), multiple ankyrin repeat domains with binding specificity for the CD3-specific binding domain of bispecific T-cell engager molecules (TCEs) were selected from DARPin® libraries in a way similar to the one described by Binz et al. 2004 (loc. cit.), with specific conditions and additional de-selection steps. The binding and specificity of the selected clones towards the CD3-specific binding domains were assessed by E. coli crude extract Homogeneous Time Resolved Fluorescence (HTRF), indicating that multiple binding proteins were successfully selected that specifically bind to the CD3-specific binding domains. These initially identified binding proteins were further developed to obtain binding proteins with even higher affinity to and/or even lower off-rate from the CD3-specific binding domains of TCEs. For example, the ankyrin repeat domains of SEQ ID NOs: 1 to 10 constitute amino acid sequences of binding proteins comprising an ankyrin repeat domain with binding specificity and high binding affinity to and/or low off-rate from the CD3-specific binding domain of bispecific T-cell engager molecules.

### CD3-Specific Binding Domains as Target and Selection Material

CD3-specific binding domains of bispecific TCEs were used as target and selection material. Such target domains were selected from the polypeptides of SEQ ID NOs: 11-15. Target proteins were biotinylated using standard methods.

### Selection of Target-Specific Ankyrin Repeat Proteins by Ribosome Display

Designed ankyrin repeat protein libraries (N2C and N3C) were used in ribosome display selections against the CD3-specific binding domain (SEQ ID NO:11) used as a target (see Binz et al., Nat Biotechnol 22, 575-582 (2004); Zahnd et al., Nat Methods 4, 269-279 (2007); Hanes et al., Proc Natl Aced Sci USA 95, 14130-14135 (1998)).

Four selection rounds were performed per target and library. The four rounds of selection employed standard ribosome display selection, using decreasing target concentrations and increasing washing stringency to increase selection pressure from round 1 to round 4 (Binz et al. 2004, loc. cit.). The number of reverse transcription (RT)-PCR cycles after each selection round was continuously reduced, adjusting to the yield due to enrichment of binders. The 3 resulting pools were then subjected to a binder screening.

### Selected Clones Bind Specifically to the CD3-Specific Binding Domain of a TCE as Shown by Crude Extract HTRF

Individually selected ankyrin repeat proteins specifically binding to the CD3-specific binding domain of a TCE in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into a derivative of the pQE30 (Qiagen) expression vector in a format, in which the clones were covalently linked to a human serum albumin (HSA)-binding domain and a CD3-binding domain (SEQ ID NO: 11), transformed into E. coli XL1-Blue (Stratagene), plated on LB-agar (containing 1% glucose and 50 μg/ml ampicillin) and then incubated overnight at 37° C. Single colonies were picked into a 96 well plate (each clone in a single well) containing 165 μl growth medium (LB containing 1% glucose and 50 μg/ml ampicillin) and incubated overnight at 37° C., shaking at 800 rpm. 150 μl of fresh LB medium containing 50 μg/ml ampicillin was inoculated with 8.5 μl of the overnight culture in a fresh 96-deep-well plate. After incubation for 120 minutes at 37° C. and 850 rpm, expression was induced with IPTG (0.5 mM final concentration) and continued for 6 hours. Cells were harvested by centrifugation of the plates, supernatant was discarded and the pellets were frozen at −20° C. overnight before resuspension in 10 μl μl B-PERII (Thermo Scientific) and incubation for one hour at room temperature with shaking (600 rpm). Then, 160 μl PBS was added and cell debris was removed by centrifugation (3220 g for 10 min).

The extract of each lysed clone was applied as a 1:800 dilution (final concentration) in PBSTB (PBS supplemented with 0.1% Tween 20® and 0.1% (w/v) BSA, pH 7.4) together with 12.5 nM (final concentration) biotinylated CD3 binding domain, 1:300 (final concentration) of anti-FLAG-D2 HTRF antibody—FRET acceptor conjugate (Cisbio) and 1:300 (final concentration) of anti-strep-Tb antibody FRET donor conjugate (Cisbio, France) to a well of a 384-well plate and incubated for 120 minutes at 4° C. The HTRF was read-out on a Tecan M1000 using a 340 nm excitation wavelength and a 620±10 nm emission filter for background fluorescence detection and a 665±10 nm emission filter to detect the fluorescence signal for specific binding.

The same lysate was mixed with 12.5 nM (final concentration) biotinylated HSA, 1:300 (final concentration) of anti-FLAG-D2 HTRF antibody—FRET acceptor conjugate (Cisbio) and 1:300 (final concentration) of anti-strep-Tb antibody FRET donor conjugate (Cisbio, France) to a well of a 384-well plate and incubated for 120 minutes at 4° C. The HTRF was read-out on a Tecan M1000 using a 340 nm excitation wavelength and a 620±10 nm emission filter for background fluorescence detection and a 665±10 nm emission filter to detect the fluorescence signal for specific binding.

The extract of each lysed clone was tested for inhibition of binding to the biotinylated CD3 bindingtarget domain, and unimpeded binding to the biotinylated HSA, in order to assess specific binding to the CD3 binding domain.

### Further Analysis and Selection of Binding Proteins with Very High Affinity for Target Protein

A total of 744 binding proteins were initially identified. Based on binding profiles, 172 candidates were selected to be expressed in 96-well format and purified to homogeneity in parallel to DNA sequencing. Candidates were characterized biophysically by size exclusion chromatography, Sypro-Orange thermal stability assessment (see Niesen et al., Nat Protoc 2, 2212-2221, (2007)), ProteOn surface plasmon resonance (SPR) target affinity assessment, ELISA, target protein-competition HTRF experiments, and/or SDS-PAGE.

In further development of the initially identified binding proteins, binders with very high affinity to and/or very low off-rate from target protein were generated using two different approaches. In the first approach, one initially identified binding protein (the “parental” binding protein) was selected as a suitable starting point for affinity maturation. The affinity maturation procedure entailed saturation mutagenesis of each randomized position of the ankyrin repeat domain used as a starting point. Sequences generated by the affinity maturation procedure were screened for lower off-rates by competition HTRF. Beneficial mutations identified thereby were combined in the binding proteins by protein engineering. The binding properties of affinity matured and engineered binding proteins were validated by surface plasmon resonance (SPR). Binders #1 to #4 (SEQ ID NOs: 1 to 4) were generated by this method, derived from the parental Binder.

In the second approach, the selection pool obtained after four rounds of ribosome display selection against the target protein (SEQ ID NO: 11) was used as a starting point for additional off-rate selections. Off-rate selections from the pool were performed consecutively against two further biotinylated target proteins (SEQ ID NO: 13 and SEQ ID NO: 14), each time in presence of an excess of the respective non-biotinylated target protein. In some instances, the off-rate selection process was combined with error-prone PCR. Several hundred binding proteins generated by this procedure were then evaluated for their binding properties by SPR.

Based on these second approach, Binders #5 to #10 (SEQ ID NOs: 5 to 10), binding to the CD3-specific binding target domains with high affinity and/or low off-rate, were chosen for further analysis. Taken together, these 10 binding proteins (SEQ ID NOs: 1 to 10) derived by these two different approaches constitute binding moieties of the invention.

These chosen binding proteins with binding specificity for a TCE CD3 binding domain were cloned into a pQE (QIAgen, Germany) based expression vector providing an N-terminal His-tag (SEQ ID NO: 20) to facilitate simple protein purification as described below. Expression vectors encoding the following binders were constructed:
Binder #1 (SEQ ID NO:1 with a His-tag (SEQ ID NO:20) fused to its N terminus);
Binder #2 (SEQ ID NO:2 with a His-tag (SEQ ID NO:20) fused to its N terminus);
Binder #3 (SEQ ID NO:3 with a His-tag (SEQ ID NO: 20) fused to its N terminus);
Binder #4 (SEQ ID NO:4 with a His-tag (SEQ ID NO: 20) fused to its N terminus);
Binder #5 (SEQ ID NO:5 with a His-tag (SEQ ID NO: 20) fused to its N terminus);
Binder #6 (SEQ ID NO:6 with a His-tag (SEQ ID NO: 20) fused to its N terminus);
Binder #7 (SEQ ID NO:7 with a His-tag (SEQ ID NO: 20) fused to its N terminus);
Binder #8 (SEQ ID NO:8 with a His-tag (SEQ ID NO: 20) fused to its N terminus);
Binder #9 (SEQ ID NO:9 with a His-tag (SEQ ID NO: 20) fused to its N terminus); and
Binder #10 (SEQ ID NO:10 with a His-tag (SEQ ID NO: 20) fused to its N terminus).

### High Level and Soluble Expression of the Binding Proteins Chosen for Analysis

For further analysis, the binders were expressed in E. coli cells and purified using their His-tag according to standard protocols. 50 ml of stationary overnight cultures (TB, 1% glucose, 50 mg/l of ampicillin; 37° C.) were used to inoculate 1000 ml cultures (TB, 50 mg/l ampicillin, 37° C.). At an absorbance of 1.0 to 1.5 at 600 nm, the cultures were induced with 0.5 mM IPTG and incubated at 37° C. for 4-5 h while shaking. The cultures were centrifuged, and the resulting pellets were re-suspended in 25 ml of TBS₅₀₀ (50 mM Tris-HCl, 500 mM NaCl, pH 8) and lysed (sonication or French press). Following the lysis, the samples were mixed with 50 KU DNase/ml and incubated for 15 minutes prior to a heat-treatment step for 30 minutes at 62.5° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art. Highly soluble ankyrin repeat proteins with binding specificity for TCE CD3 binding domain were purified from E. coli culture (up to 200 mg ankyrin repeat protein per litre of culture) with a purity >95% as estimated from 4-12% SDS- PAGE.

### Example 2: SPR Binding Assays

An important feature of a binding moiety of the invention is its affinity towards a drug molecule. Relevant aspects include the off-rate of the binding moiety from the drug molecule and the resulting blocking half-life. A high affinity, low off-rate and significant blocking half-life are required to achieve slow release of the drug molecule from a complex, in which the drug molecule is reversibly bound by a binding moiety. See FIGS. 1 to 3 for further illustration and explanation of function and properties of prodrug complexes of the invention.

Surface plasmon resonance (SPR) assays were used to determine the binding affinity and off-rate of ankyrin repeat binding domains to the CD3 binding domain of a TCE drug molecule.

All SPR data were generated using a Bio-Rad ProteOn XPR36 instrument with PBS-T (0.005% Tween 20) as running buffer. A new neutravidin sensor chip (NLC) was air-initialized and conditioned according to Bio-Rad manual.

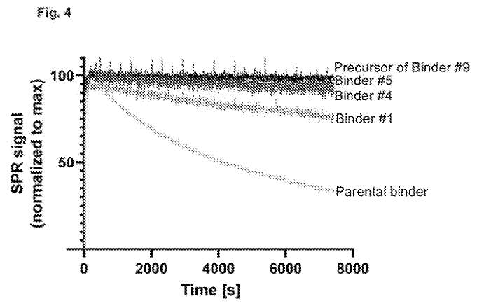

SPR data were generated for Binders #1, #4, #5 and a precursor of Binder #9 (as listed in Example 1 above), as well as for the parental binder used for the affinity maturation approach (see Example 1), binding to biotinylated CD3-specific binding domains having SEQ ID NOs: 13 and 14, respectively, on the chip. The data were generated at 25° C. and with one binder concentration of 100 nM (i.e. single-trace) measuring the off-rate during 2 h. The SPR traces obtained with SEQ ID NO: 13 as target protein are shown in FIG. 4. Binders #1, #4, #5 and the precursor of Binder #9 all displayed strongly reduced off-rates compared to the parental binder. Similar results were obtained with SEQ ID NO: 14 as a target protein.

Further SPR data were generated for Binders #1 to #9 (as listed in Example 1 above), binding to biotinylated CD3-specific binding domains having SEQ ID NOs: 13 and 14, respectively, on the chip. The data were generated at 33° C. and with several binder concentrations (26.7 nM, 3 nM, 1 nM) (i.e., multi-trace) measuring on-rate (kₒₙ), the off-rate (k_{off}) during 2 h and deriving the equilibrium dissociation constant (K_{D}). The blocking half-life (T_{1/2}) was then calculated. Results are provided in Table 2 below:

TABLE 2

| | Biotinylated CD3-specific binding domain (Ligand): | | | | | | Biotinylated CD3-specific binding domain (Ligand): | | | | | |
|---|---|---|---|---|---|---|---|---|---|---|---|---|
| | SEQ ID NO: 13 | | | | | | SEQ ID NO: 14 | | | | | |
| Binder | | | | Blocking | | | | | | Blocking | | |
| name | kₒₙ | k_{off} | KD | T½ | Rmax | Chi2 | kₒₙ | k_{off} | KD | T½ | Rmax | Chi2 |
| (Analyte) | [×10⁵ M⁻¹ s⁻¹] | [×10⁻⁵ s⁻¹] | [pM] | [h] | [RU] | [RU] | [×10⁵ M⁻¹ s⁻¹] | [×10⁻⁵ s⁻¹] | [pM] | [h] | [RU] | [RU] |
| Binder #1 | 15.4 | 6.2 | 40 | 3.1 | 113.7 | 6.4 | 7.9 | 2.7 | 34 | 7.1 | 96.3 | 5.3 |
| Binder #2 | 14.3 | 4.0 | 28 | 4.8 | 117.4 | 4.9 | 6.3 | 2.0 | 32 | 9.6 | 132.3 | 6.9 |
| Binder #3 | 17.0 | 2.8 | 16 | 6.9 | 117.0 | 4.6 | 6.5 | 1.9 | 29 | 10.1 | 139.1 | 6.5 |
| Binder #4 | 17.4 | 1.9 | 11 | 10.1 | 107.6 | 10.4 | 6.3 | 1.2 | 19 | 16.0 | 136.7 | 6.1 |
| Binder #5 | n.d. | <5 | <20 | >3.9 | n.d. | n.d. | n.d. | <5 | <20 | >3.9 | n.d. | n.d. |
| Binder #6 | n.d. | <5 | <20 | >3.9 | n.d. | n.d. | n.d. | <5 | <20 | >3.9 | n.d. | n.d. |
| Binder #7 | n.d. | <5 | <20 | >3.9 | n.d. | n.d. | n.d. | <5 | <20 | >3.9 | n.d. | n.d. |
| Binder #8 | n.d. | <5 | <20 | >3.9 | n.d. | n.d. | n.d. | <5 | <20 | >3.9 | n.d. | n.d. |
| Binder #9 | n.d. | <5 | <20 | >3.9 | n.d. | n.d. | n.d. | <5 | <20 | >3.9 | n.d. | n.d. |

Table 2 provides kₒₙ, k_{off} and K_{D} values of some super-high affinity binders (analytes) against two different CD3 binding proteins (SEQ ID NO: 13 and SEQ ID NO: 14), with the blocking T_{1/2} calculated from the off-rate (k_{off}) using the formula T_{1/2}=ln2/k_{off}=0.693/k_{off}. The off-rates of the binders were all below 1×10⁻⁴ s⁻¹. The K_{D} values were all below 1×10⁻¹⁰ M. SPR measurements for Binders #5 to #9 (SEQ ID NOs: 5 to 9) did not yield more precise values than those shown in Table 2 due to the lack of signal drop during dissociation phase (on which koff calculation is based) of at least 10%.

All binders displayed a blocking half-life (T_{1/2}) of at least 3 hours, with some of them displaying a blocking half-life of at least 10 hours. Thus, these experiments showed that Binder #1 to #9 have very high binding affinity to CD3-specific binding domains, which are used in TCE drug molecules. Furthermore, Binder #1 to #9 have very low off-rates from these target proteins and hence very significant blocking half-lives. Binder #10 displays similar properties as Binder #9 when tested in comparable SPR assays.

### Example 3: T-Cell Activation/Killing Assay

In this example, three binding moieties of the invention and the parental binder were tested for their potential to inhibit T-cell activation by a T-cell engager (called TCE #1 herein) and killing of tumor cells by activated T-cells. TCE #1 comprises a CD3-specific binding domain (SEQ ID NO: 13 covalently connected to a tumor-associated antigen (TAA)-specific binding domain (TAA binding domain). TAA is expressed on the tumor cells used for the assay.

Prodrug complex samples were prepared as indicated below, at 100× higher concentrations compared to the final assay concentration, in order to ensure that substantially all TCE is bound by the binding moieties.

### Preparation of Prodrug Complex Samples and Addition to T-Cell Activation/Killing Assay

1. Prepare TCE constant at final 10 pM 200× concentrated (2 nM)
2. Prepare binding moiety titration with final start 10 nM, 200× concentrated (start 2000 nM, 1:3 serial dilution: 2000 nM, 666 nM, 222 nM, 74 nM, 25 nM, 8.2 nM, 2.7 nM, 0.91 nM, 0.30 nM)
3. Mix TCE with binding moiety titration 1:1 (or TCE alone or binding moiety alone with assay media)→100×
4. Equilibrate for at least 24 h in pH-controlled incubator, 37° C.
5. Dilute into T-cell activation/killing experiment right before assay start 1:100 (2 μl in 200 μl Volume)→1×
6. Incubate for 48 h

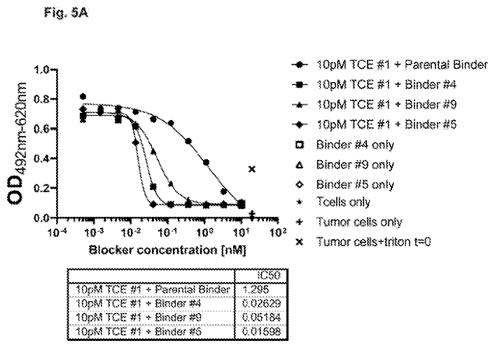

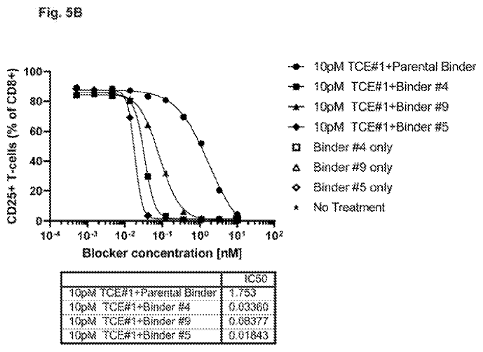

For the T-cell activation assay, target tumor cells and effector T-cells (pan T-cells from healthy blood donors) were combined at an effector to target cell ratio of 5:1, prodrug complex samples were added, and the mixtures were incubated for 48 hours at 37° C. Supernatant was analyzed for LDH release of killed tumor cells and the levels of activation markers (CD25) on CD8⁺ T-cells were determined by FACS (using CD25 Monoclonal Antibody (BC96), PerCP-Cyanine5.5, eBioscience™). Various controls were included as indicated in FIGS. 5a and 5b (i.e., T cells only, tumor cells only, Triton control, binding moieties only).

Results are provided in FIGS. 5a and 5b. It can be seen that the three binding moieties of the invention (Binder #4 (SEQ ID NO: 4); Binder #5 (SEQ ID NO: 5); and Binder #9 (SEQ ID NO: 9) inhibited T-cell activation and tumor cell killing at much lower concentrations than the parental binder, consistent with the much higher binding affinity of the binding moieties of the invention. IC50 values (in nM) are provided in FIGS. 5a and 5b.

### Example 4: “Simple” Incucyte® Assays

Standard T-cell activation assays, such as those in Example 3 above, provide a single readout after a defined timepoint. For the purposes of the present invention, multiple readouts at different timepoints are desired to obtain a time-resolved view on T-cell mediated tumor cell growth inhibition, used as a surrogate of T-cell mediated tumor cell killing. Incucyte® assays (see https://www.essenbioscience.com/en/products/incucyte/) can provide such information. The Incucyte assay principle is that T-cells (either pan-T-cells of hPBMCs) are co-incubated with tumor cells, which have been transduced with NucLight Red Lentivirus that renders the cells red and thus detectable for the Incucyte camera, and that by determining the area of red objects (i.e. tumor cells that are labelled by Incucyte Nulight®, a nuclear-restricted mKate2 protein (red fluorescent protein)) the tumor cell growth in the presence of T cells can be monitored by the Incucyte camera in a time-resolved fashion.

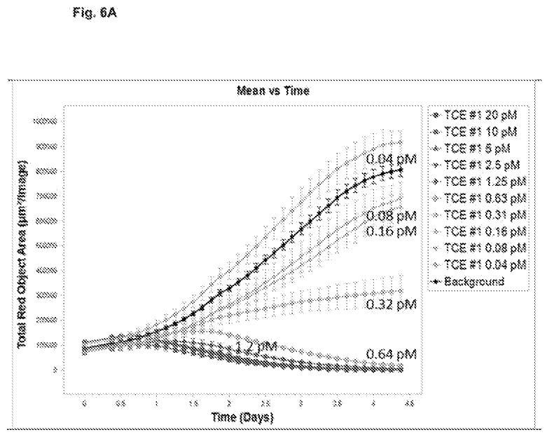

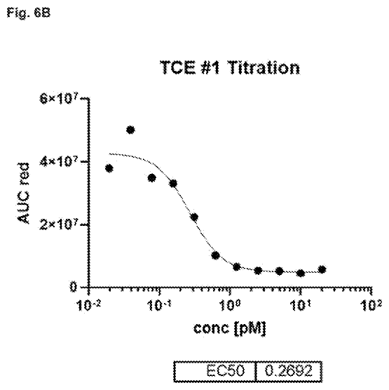

In an initial experiment, the T-cell engager (TCE #1) was prepared 100-fold concentrated in a 2-fold serial dilution, starting from a concentration of 2 nM (20 pM final concentration in the assay). TCE #1 was added to pan T-cells and tumor cells expressing the TAA and labelled with NucLight Red for detection by Incucyte®. The growth of red-labelled tumor cells was observed in the Incucyte® device over the course of 4.5 days. FIG. 6a shows that at higher concentrations of TCE #1 (starting from 20 pM) TCE #1 efficiently inhibited tumor cell growth and that with decreasing concentrations (in 2-fold steps) the inhibition of tumor cell growth was gradually reduced, with a transition between 0.16 and 0.64 pM (EC₅₀ of about 0.27 pM). Thus, below 0.1 pM the TCE had no significant impact on tumor cell growth, whereas between 0.1 and 1 pM the TCE was in the dynamic range of T-cell mediated tumor cell growth inhibition, and whereas at >1 pM the TCE led to full T-cell mediated tumor cell growth inhibition (used as a surrogate of T-cell mediated tumor cell killing). FIG. 6b illustrates these findings and shows that the EC50 of TCE activity was at about 0.27 pM.

### “Simple” Incucyte® Assay Setup with Titration of Prodrug Complex

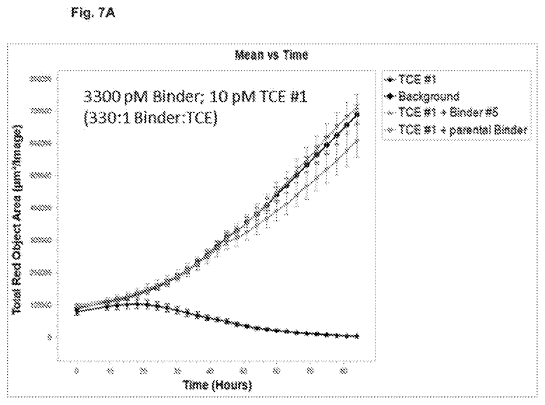

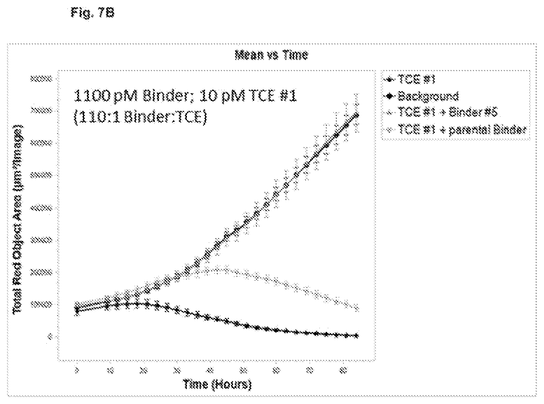

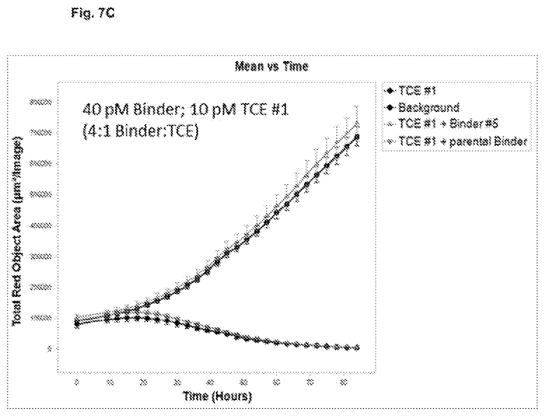

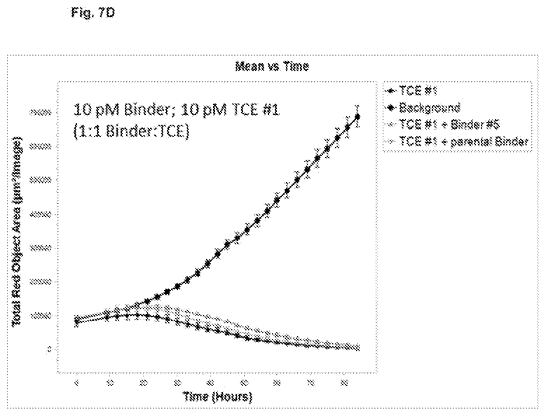

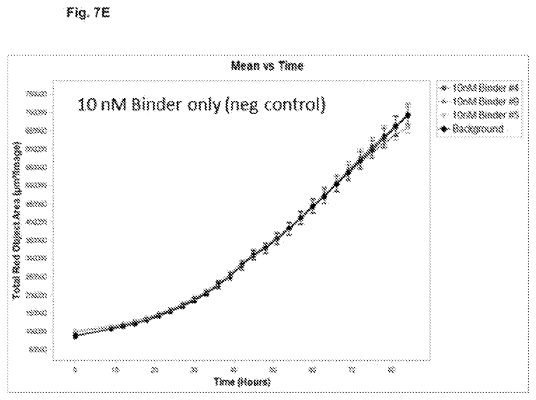

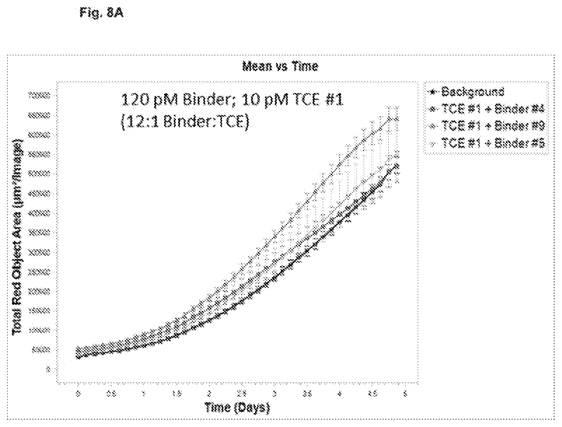

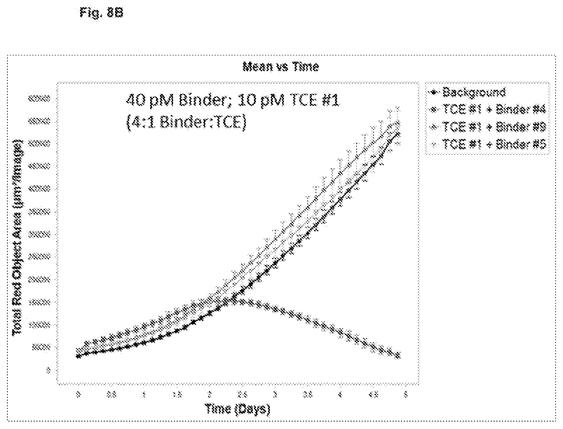

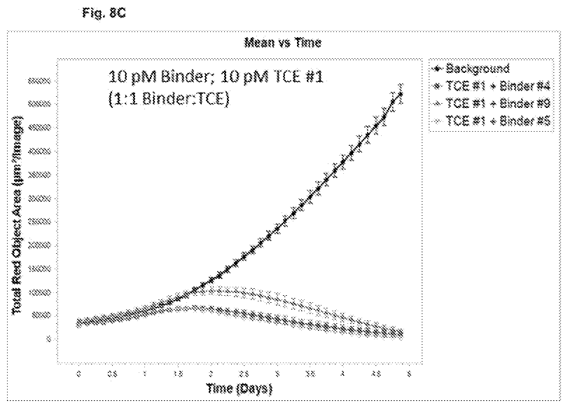

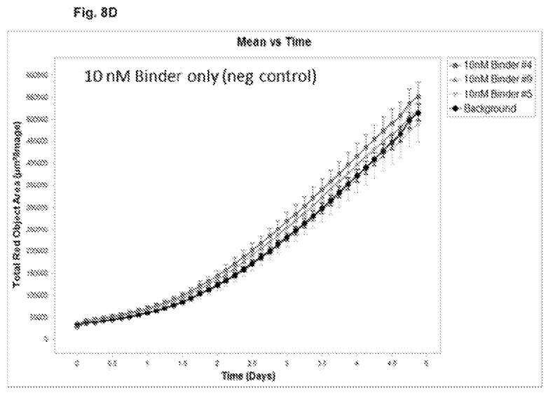

In order to investigate the effect of binding moieties of the invention on TCE-mediated inhibition of TAA-expressing tumor cell growth, a further Incucyte® assay was performed with various drug complex samples. Drug complex samples were prepared with the titration of binding moieties to a constant concentration of TCE (TCE #1). The mixtures were prepared as a 100× concentrated stock to ensure that substantially all of the drug molecules were complexed by the binding moieties at the start of the experiment (i.e., the addition of prodrug complex to the T-cells and tumor cells). The equilibrated 100× concentrated stock was diluted 100-fold by adding it to the cells, and the change in growth of red-labelled tumor cells was followed over the duration of 4.5 days, as a surrogate for T-cell mediated tumor cell killing (see FIGS. 7 and 8).

### Preparation of Prodrug Complex Samples and Addition to Incucyte® Experiment

1. Prepare TCE constant at final 10 pM 200× concentrated (2 nM)
2. Prepare binding moiety titration with final starting concentration of 10 nM, 200× concentrated (start 2 μM)
3. Mix TCE with binding moiety titration 1:1 (or TCE alone or binding moiety alone with assay media)→100× concentrated stock sample
4. Equilibrate for at least 24 hours in pH-controlled incubator at 37° C.
5. Dilute into experiment (T-cells+tumor cells labeled with NucLight Red) right before assay start 1:100 (2 μl in 200 μl volume)
6. Determine tumor cell proliferation for up to 5 days in Incucyte®

In a first experiment, the ability of the simple Incucyte® assay to distinguish between the parental binder (K_{D}˜200 pM) and the super-high affinity binding moiety having SEQ ID NO: 5 (K_{D}˜6 pM or lower) (Binder #5) was investigated. FIGS. 7a to 7d clearly show that the parental binder exhibited the loss of blocking between 330- and 110-fold molar excess of parental binder over drug molecule in the samples, whereas the super-high affinity binding moiety (Binder #5) blocked the activity of the drug molecule fully even at the much lower 4-fold molar excess of binding moiety over drug molecule in the prodrug complex samples.

In a second experiment, different super-high affinity binding moieties were investigated in an analogous setup (FIGS. 8a to 8c). With decreasing molar excess of binding moiety over drug molecule (TCE #1) in the drug complex samples, loss of inhibition of the anti-tumor activity of the drug molecule was observed. This loss of inhibition showed a correlation with the binding affinities of the binding moieties to the drug molecule. Binder #4 showed already at 4-fold molar excess a loss of inhibition of TCE activity, leading to increased inhibition of tumor cell growth, whereas the other two binding moieties, Binder #5 and Binder #9, still effectively inhibited TCE activity at a 4-fold molar excess, resulting in normal growth curves without any T-cell mediated inhibition. Only when the molar ratio was reduced to 1:1, did Binder #5 and Binder #9 show a loss of inhibition of TCE activity, leading to increased T-cell mediated inhibition of tumor cell growth. Even at a molar ratio of 1:1, Binder #9 could inhibit TCE activity to some degree, resulting in a weak inhibition of tumor cell growth.

These data confirm that the Incucyte® assay is a sensitive method to investigate TCE blocking by super-high affinity binding moieties, and that the binding moieties exhibit blocking activity as expected from the super-high affinities shown by SPR in Example 2.

Overall, these experiments indicated that binding moieties of the invention can effectively inhibit biological activity of a drug molecule, depending on the binding affinity of the binding moiety to the drug molecule, as demonstrated here in exemplary fashion with TCE #1 as drug molecule and binders described in Example 1 as binding moieties.

### Example 5: “Complex” Incucyte® Assay

In an in vivo situation, a binding moiety is eliminated quickly as soon as it dissociates from the drug molecule due to its short half-life. In contrast, a drug molecule can circulate in the body for a much longer time, independently of whether it is complexed or not, if it has been designed to have a long half-life. Methods to generate drug molecules with a long half-life are well known in the art, involving, for example, the fusion to a half-life extending moiety. To mimic the quick renal elimination of the binder, the “complex” Incucyte® setup is equipped with an additional so-called “sink” chamber. In this sink chamber, a large excess of bead-immobilized target protein of the tested binding moiety is added (>10,000-fold molar excess over the amount of binding moiety contained in the prodrug complex added to the main chamber). This immobilized target protein scavenges the free binding moiety as soon as it dissociates from the prodrug complex and diffuses into the sink chamber.

In the experiment described here, a TCE (TCE #1) was used as drug molecule. Hence, the immobilized scavenger protein added in the sink chamber was a CD3-specific binding domain (SEQ ID NO:11) that is bound with high affinity by the tested binding moiety.

### Experimental Setup

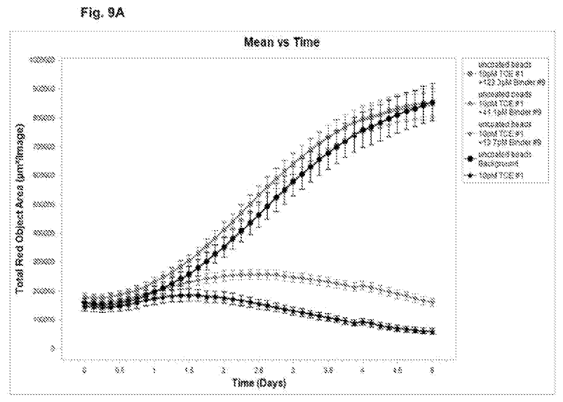

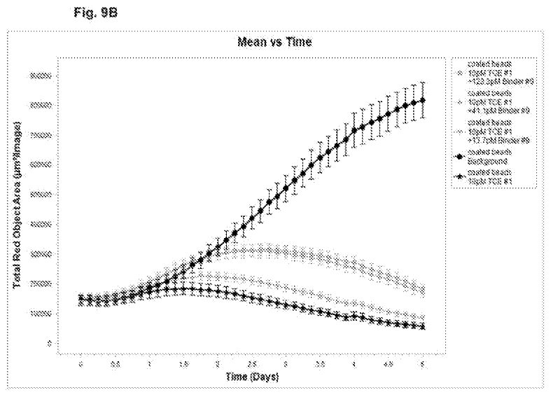

As in Example 4, a tumor cell line expressing the TAA was used as target cell line which has been transduced with Incucyte Nuclight Red Lentivirus for detection by the Incucyte® camera. PanT-cells were added, together with different prodrug complexes. The prodrug complexes were generated by pre-equilibrating 10 pM TCE #1 (final concentration) with different amounts of Binder #9, at 123 pM (12.3-fold molar excess), 41 pM (4.1-fold molar excess) and 13.7 pM (1.37-fold molar excess). Each of these prodrug complexes was then used in a “complex” Incucyte® setup with either uncoated beads that do not sequester free binding moiety (FIG. 9a) as negative control, or with CD3-specific binding domain coated beads, thus having a functional sink (FIG. 9b). Additionally, unbound TCE #1 (10 pM) as well as background (i.e. no prodrug complex or TCE, but with beads) served as controls. Tumor cell growth was measured for a period of 5 days by determining the area of red objects detected by the Incucyte® camera.

FIG. 9a shows that in wells with a sink chamber containing non-coated beads, the tested binding moiety completely inhibited TCE activity and hence no T-cell mediated inhibition of tumor cell growth was observed, except for prodrug complexes generated with the lowest binder:TCE ratio (1.37:1). This is consistent with the results shown in Example 4. In contrast, in wells with a functional sink chamber (FIG. 9b) containing beads coated with CD3-specific binding domain, the tested binding moiety inhibited TCE activity only partially and hence T-cell mediated inhibition of tumor cell growth was observed, even at the highest binder:TCE ratio (12.3:1). In contrast to the control with unbound TCE, the T-cell mediated inhibition of tumor cell growth (as a surrogate of T-cell mediated tumor cell killing) in wells with prodrug complex started with a significant delay, only about 1.5-2 days after the start of the experiment. Inhibition of tumor cell growth in wells with unbound TCE started already about 1 day after the start of the experiment. The delayed activity of the TCE drug molecule, reflected in the delayed inhibition of tumor cell growth, demonstrated that the binding moiety can delay the biological activity of the drug molecule when complexed with the drug molecule to form a prodrug complex. These data support the concept that binding moieties of the invention can form a prodrug complex with a drug molecule, resulting in slowed release of the drug molecule and hence down-modulated activity of the drug molecule upon administration of the prodrug complex to a patient, as compared to administration of the unbound drug molecule.

### Example 6: Transiently Blocking the CD3 Effector Moiety of a TCE Drug Molecule by Complexation with a Binder Reduces Cytokine Release in Ex Vivo Human Whole Blood Assay

In order to investigate the effect of a Binder on the cytokine release profile of a TCE drug molecule (in this example, TCE #1 (SEQ ID NO: 54), an α-CD123×α-CD33×α-CD3 T-cell engager), an ex vivo human whole blood assay was carried out at Immuneed AB, Sweden.

The applied whole blood loop system can be used to study interactions between blood and a drug sample, including the effect on cytokine release. The blood loop system uniquely includes both immune cells in the blood, immunoglobulins and intact complement and coagulation cascade systems (Fletcher, E. A. K., et al., Int Immunopharmacol, 2018. 54: p. 1-11.). Cytokine release was determined by spiking test articles into fresh human whole blood from one healthy human donor containing CD123+ and CD33+ target cells, and subsequently incubating the samples on a rotating wheel to avoid blood clotting and mimicking blood circulation.

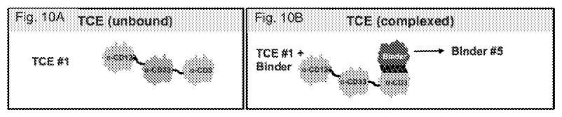

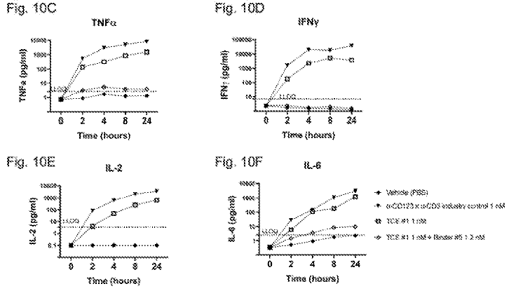

As controls, vehicle or 1 nM anti-CD123×anti-CD3 industry control test article were spiked into the human whole blood. TCE #1 molecule was spiked in at 1 nM concentration and compared to 1 nM TCE #1+1.2 nM Binder #5. Complexed TCE was pre-equilibrated with a 1.2-fold molar excess of Binder to guarantee 100% complexation rate of the TCE at the start of the experiment. Test articles are shown in FIG. 10A and 10B.

Cytokine release for human TNF-α, IFN-γ, IL-2 and IL-6 was determined by Meso Scale MULTI-ARRAY® technology at time-points 0 h, 2 h, 4 h, 8 h, 24 h.

As shown in FIGS. 10 C-F, increasing levels of all cytokines over vehicle were observed in response to α-CD123×α-CD3 industry control and, to a lesser extent, unbound TCE #1 during the 24 h measured. However, the complexed TCE consisting of TCE #1 complexed with Binder #5 greatly reduced cytokine release for TNF-α, IFN-γ, IL-2 and IL-6 compared to unbound TCE #1 at all time-points.

In conclusion, TCE prodrug complex comprising a TCE (such as TCE #1) in complex with a Binder (such as Binder #5) was shown to effectively suppress cytokine release in an ex vivo human whole blood assay.

### Example 7: TCE Prodrug Complex Maintains Optimal Anti-Tumor Activity in an In Vivo Efficacy Study

The aim of the in vivo efficacy study was to compare the anti-tumor activity of an unbound TCE drug molecule (in this example, TCE #2 (SEQ ID NO: 55), an α-HSA×α-CD123×α-CD33×α-CD3 T-cell engager comprising a half-life extending moiety (α-HSA)) and a complexed TCE drug molecule (a TCE prodrug complex) (in this example, TCE #2 in complex with one of two different Binders having affinities either in the double-digit pM (Binder #4) or even 1 pM or lower range (Binder #10)). NOG mice were humanized on d0 with PBMCs from two human donors (n=5 donor A and n=5 donor B). 1×10⁶ Molm-13 tumor cells were injected subcutaneously on d2 and treatment was initiated on d6 after randomization at tumor sizes ˜70 mm³. Quality control included analysis of the PBMC subpopulations and viability by FC one day after isolation, as well as successful humanization of mice blood by FC on d19. Molm-13 cells were tested for viability and CD33 positivity one day before injection.

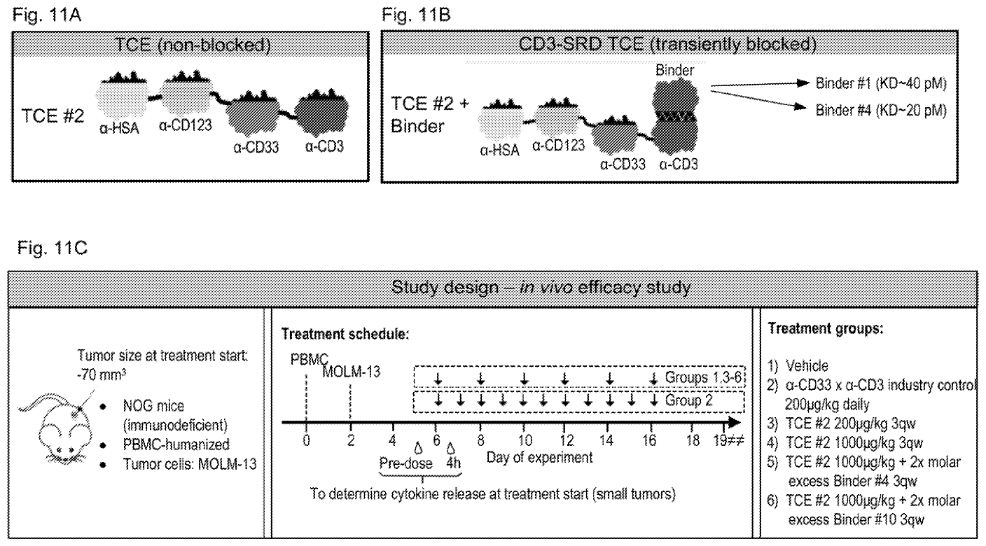

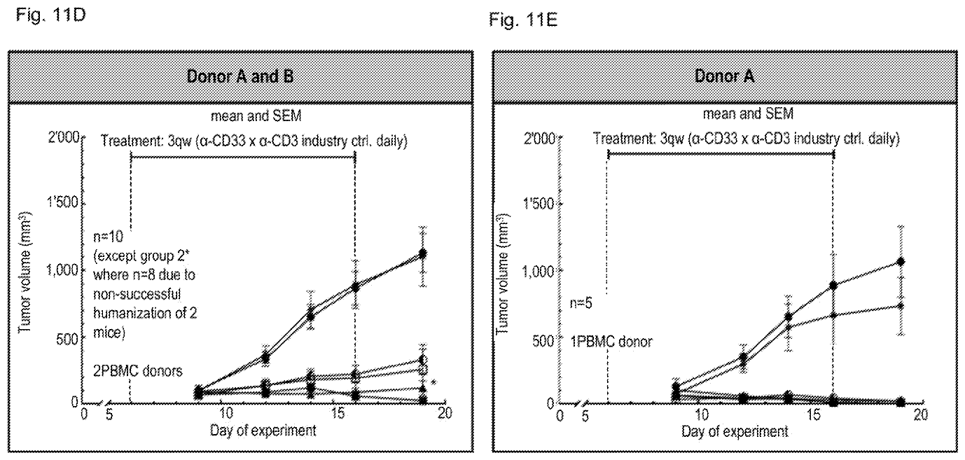

Test articles for the in vivo efficacy study are shown in FIGS. 11A and 11B. The study design and treatment groups are outlined in FIG. 11C. The TCE #2 was given at either 200 μg/kg or 1000 μg/kg dose and was compared to TCE #2 at 1000 μg/kg in complex with Binder #4 (K_{D}˜20 pM) or Binder #10 (K_{D}≤1 pM). Complexes were pre-equilibrated using a 2× molar excess of Binder to guarantee 100% complexation rate at treatment start. Treatment was given i.v. 3qw from d6 to d16 for all treatment groups including vehicle, except for the anti-CD33×anti-CD3 industry control group 2 where treatment was given daily at 200 μg/kg. Blood samples were taken prior and 4 h post first treatment dose in order to determine cytokine levels at treatment start with these relatively small tumors. Tumor growth was monitored and measured 3 times weekly with a calliper in width and length of which the tumor volume was calculated using the formula length×width×height×π/6.

Tumor growth curves for all six treatment groups plotted as mean±SEM for n=10 mice humanized either with PBMCs of Donor A or B are shown in FIG. 11D. For group 2 (α-CD33×α-CD3 industry control) two animals humanized with PBMCs from Donor B were excluded from the analysis due to non-successful humanization.

For donor A and B combined, strong tumor growth inhibition was observed for the non-half-life extended anti-CD33×anti-CD3 industry control group given at 200 μg/kg daily, and tumor eradication was observed for the half-life-extended TCE # 2 given at 1000 μg/kg 3qw. A less pronounced tumor growth inhibition was observed for TCE # 2 given at 200 μg/kg 3qw, as well as for the TCE prodrug complex comprising 1000 μg/kg TCE #2+2× molar excess of Binder #4, dosed 3qw. Similar tumor growth inhibition was not observed under these experimental conditions for the TCE prodrug complex comprising 1000 μg/kg TCE #2+2× molar excess of Binder #10.

In silico modeling predicts similar AUC between 200 μg/kg free TCE and 1000 μg/kg TCE-Binder complex consisting of TCE #2 transiently blocked with Binder #4 (K_{D}˜20 pM). In contrast, the combination of the PK of TCE #2 and the very high affinity of Binder #10 (K_{D}≤1 pM) is expected to result in lower exposure of active TCE, as a larger proportion of the TCE #2+Binder #10 complex may be eliminated from circulation before the Binder can release the active TCE #2. Thus, the “sweet spot” in terms of optimal reduction of cytokine release while maintaining full anti-tumor efficacy is an interplay of Binder affinity and the serum half-life of the TCE. The presented slow release TCE prodrug toolbox allows to match an adequate Binder to a TCE.

FIG. 11E shows tumor growth curves for all six treatment groups plotted as mean±SEM for n=5 mice humanized with PBMCs of Donor A, while FIG. 11F shows tumor growth curves for all six treatment groups plotted as mean±SEM for n=5 mice humanized with PBMCs of Donor B, except for group 2 where two animals were excluded due to non-successful humanization. A pronounced donor-to-donor variability was observed in this experiment, with PBMCs from donor A showing a stronger inhibitory effect on the growth of Molm-13 cells than PBMCs from donor B. FIG. 11G shows the legend for FIGS. 11D-F.

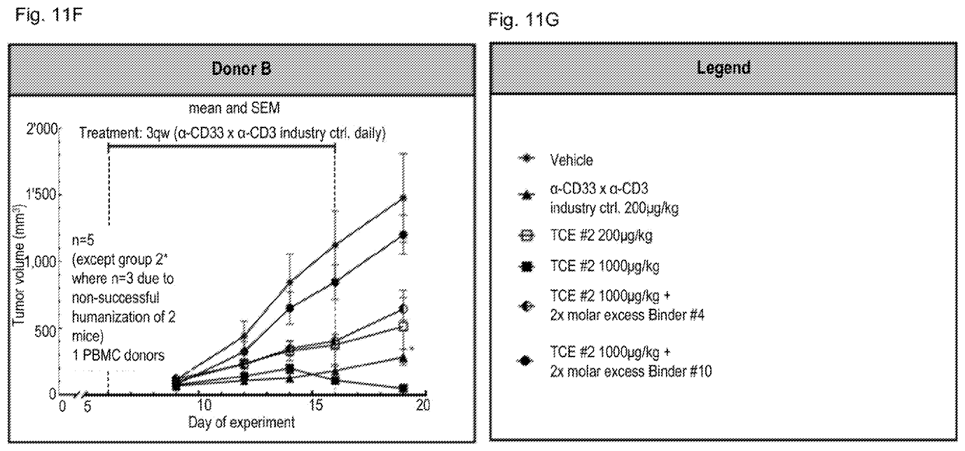

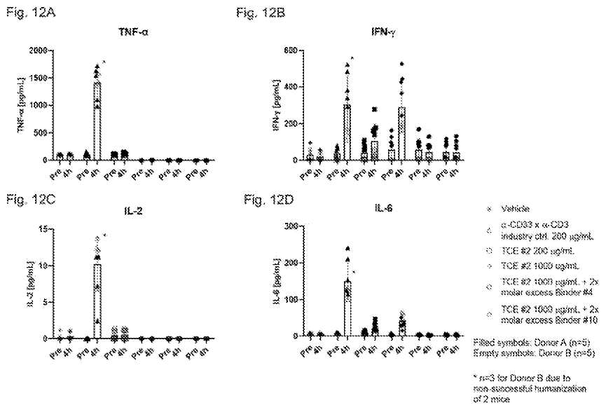

FIG. 12 shows cytokine levels determined in serum of blood samples taken prior and 4 h post first treatment dose of the in vivo efficacy study, when tumors were still relatively small. Human cytokine levels were determined on undiluted serum samples by CBA human Th1/Th2/Th17 kit (BD Biosciences).

A marked increase in cytokines from 0 h to 4 h can be observed for all measured cytokines (TNF-α, IFN-γ, IL-2 and IL-6) for the α-CD33×α-CD3 industry control and to a lesser extent for the TCE #2 at both doses administered. This increase in cytokines is however prevented with the TCE prodrug complex comprising TCE #2+Binder #4 or Binder #10, supporting the finding that complexation of a TCE drug molecule with a high affinity Binder leads to a slow release of active TCE and thus an altered exposure profile.

In summary, a slow release TCE prodrug complex (comprising TCE #2 in complex with Binder #4 (K_{D}˜20 pM to the CD3 effector moiety)) showed equivalent anti-tumor efficacy to the unbound TCE at lower dose and reduced the mild cytokine levels of TCE #2 4 h post the first treatment dose.

### Example 8: Slow Release TCE Prodrug Complex Reduces Cytokine Release in In Vivo Safety Studies

The aim of two independent in vivo safety studies was to compare the extent of cytokine release triggered by an unbound TCE drug molecule (in this example, TCE #2, an α-HSA×α-CD123×α-CD33×α-CD3 T-cell engager) and a slow release TCE prodrug complex (in this example, TCE #2 in complex with one of three different Binders having affinities in the double-digit pM (Binder #1 and Binder #4, study 1) or even 1 pM range (Binder #10, study 2)).

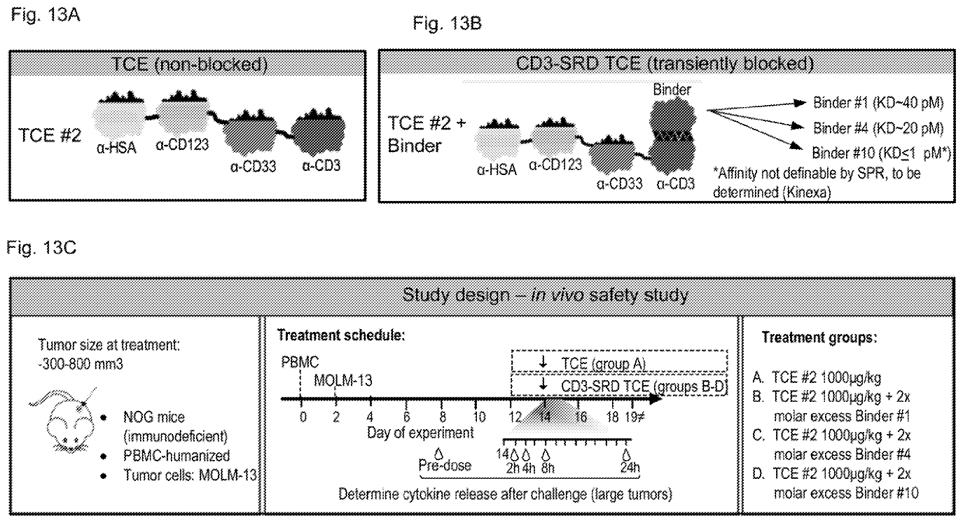

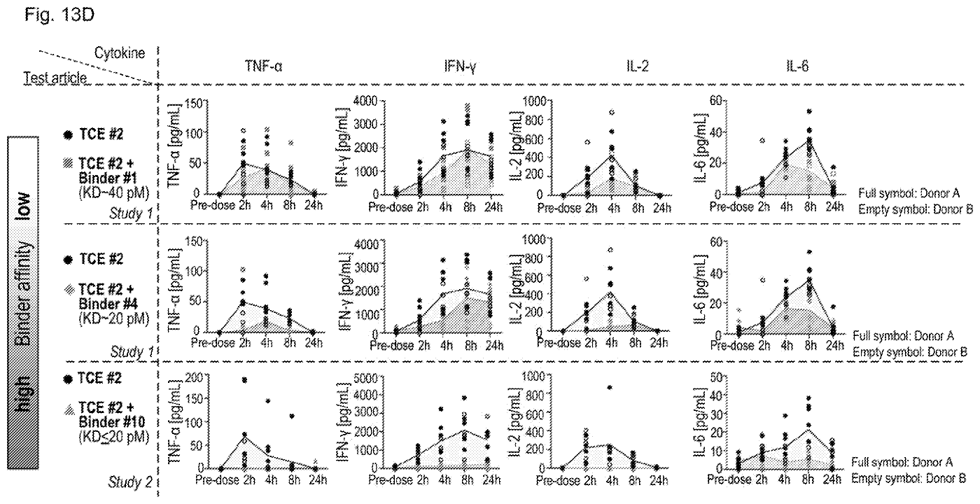

Test articles for the in vivo safety study are shown in FIGS. 13A and 13B. The study design and treatment groups are outlined in FIG. 13C. The reason to de-couple the in vivo safety studies from the in vivo efficacy study was the requirement for mid-sized tumors in order to have a sufficient amount of target cells and thus elicit a measurable cytokine response upon first administration of a TCE.

NOG mice were humanized on dO with PBMCs from two human donors (n=5 donor A and n=5 donor B). 1×10⁶ Molm-13 tumor cells were injected subcutaneously on d2, and a single dose (challenge) was given intravenously on d14 at tumor sizes ˜300-800 mm³. Quality control included analysis of the PBMC subpopulations and viability by FC one day after isolation, as well as successful humanization of mice blood by FC on d15 (study 1) or d19 (study 2). Half-life extended unbound TCE #2 administered at 1000 μg/kg was compared to TCE prodrug complex (TCE #2 in complex with Binder #1, Binder #4 or Binder #10). Complexes were pre-equilibrated with a 2-fold molar excess of Binder to guarantee 100% complexation rate at treatment start. Blood samples were taken pre-dose, 2 h, 4 h, 8 h and 24 h post first treatment dose in order to determine cytokine levels with mid-size tumors. FIG. 13D shows human cytokine levels (TNF-α, IFN-γIL-2 and IL-6) in serum from blood samples taken prior or at the indicated time-points post the single dose treatment. Cytokine levels were determined on undiluted serum samples by CBA human Th1/Th2/Th17 kit (BD Biosciences).

All cytokines are elevated after a single dose of unbound TCE #2, while reduced cytokine release can be observed for the three different TCE prodrug complexes tested. The reduction in cytokine release indeed correlates with Binder affinity and becomes more pronounced as Binder affinity increases from top row (Binder #1 with K_{D}˜40 pM) to middle row (Binder #4 with K_{D}˜20 pM) to bottom row (Binder #10 with K_{D}≤1 pM). Binder #10 almost completely inhibited cytokine release.

In conclusion, slow release TCE prodrug complexes were able to reduce cytokine release in vivo compared to unbound TCE. Moreover, Binder affinity to the CD3 effector moiety in the TCE correlated with the reduction in cytokine release.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific embodiments of the invention described herein. Such equivalents are intended to be encompassed by the following claims.

Sequences

| | Name | | |
|---|---|---|---|
| | of the | | |
| SEQ | His- | | |
| ID | tagged | | |
| NO | version | Description | Sequence |
| 1 | Binder | Designed ankyrin | DLGKKLLQAARAGQLDEVRELLKAGADVNAKDFKGLTPLHLA |
| | #1 | repeat domain | AAHGHLEIVEVLLKAGADVNAKDVYGWTPLHIAAASGHLEIVE |
| | | | VLLKAGADVNAKDWLGITPLHLAASHGHLEIVEVLLKAGADVN |
| | | | AQDKSGKTPADLAARAGHQDIAEVLQKAA |
| 2 | Binder | Designed ankyrin | DLGKKLLQAARAGQLDEVRELLKAGADVNAKDFKGLTPLHLA |
| | #2 | repeat domain | AAHGHLEIVEVLLKAGADVNAKDVYGWTPLHWAAASGHLEIV |
| | | | EVLLKAGADVNAKDWLGITPLHLAASHGHLEIVEVLLKAGADV |
| | | | NAQDKSGKTPADLAARAGHQDIAEVLQKAA |
| 3 | Binder | Designed ankyrin | DLGKKLLQAARAGQLDEVRELLKAGADVNAKDFKGLTPLHLA |
| | #3 | repeat domain | AAHGHLEIVEVLLKAGADVNAKDVYGWTPLHWAAAKGHLEIV |
| | | | EVLLKAGADVNAKDWLGITPLHLAASHGHLEIVEVLLKAGADV |
| | | | NAQDKSGKTPADLAARAGHQDIAEVLQKAA |
| 4 | Binder | Designed ankyrin | DLGKKLLQAARAGQLDEVRELLKAGADVNAKDFKGLTPLHLA |
| | #4 | repeat domain | AEHGHLEIVEVLLKAGADVNAKDVYGWTPLHWAAAKGHLEIV |
| | | | EVLLKAGADVNAKDWLGITPLHLAASHGHLEIVEVLLKAGADV |
| | | | NAQDKSGKTPADLAARAGHQDIAEVLQKAA |
| 5 | Binder | Designed ankyrin | DLGKKLLQAARAGQLDEVRELLKAGADVNAKDHYGWTPLHI |
| | #5 | repeat domain | AAQIGHLEIVEVLLKAGADVNAKDWIGYTPLHLAASHGHLEIV |
| | | | EVLLKAGADVNAKDVSGKTPLHVAAAHGHLEIVEVLLKAGAD |
| | | | VNAQDKSGKTPADLAARAGHQDIAEVLQKAA |
| 6 | Binder | Designed ankyrin | DLGKKLLQAARAGQLDEVRELLKAGADVNAKDQYGWTPLHL |
| | #6 | repeat domain | AAYSGHLEIVEVLLKAGADVNAKDWVGWTPLHLAASHGHLEI |
| | | | VEVLLKAGADVNAKDEAGRTPLHIAAKQGHLEIVEVLLKAGAD |
| | | | VNAQDKSGKTPADLAARAGHQDIAEVLQKAA |
| 7 | Binder | Designed ankyrin | DLGKKLLQAARAGQLDEVRELLKAGADVNAKDHYGWTPLHL |
| | #7 | repeat domain | AAAEGHLEIVEVLLKAGADVNAKDWIGYTPLHIAASHGHLEIV |
| | | | EVLLKAGADVNAKDSSGKTPLHIAAQHGHLEIVEVLLKAGADV |
| | | | NAQDKSGKTPADLAARAGHQDIAEVLQKAA |
| 8 | Binder | Designed ankyrin | DLGKKLLQAARAGQLDEVRELLKAGADVNAKDHYGWTPLHI |
| | #8 | repeat domain | AAQRGHLEIVEVLLKAGADVNAKDWLGWTPLHVAASHGHLEI |
| | | | VEVLLKAGADVNAKDLSGRTPLHIAARQGHLEIVEVLLKAGAD |
| | | | VNAQDKSGKTPADLAARAGHQDIAEVLQKAA |
| 9 | Binder | Designed ankyrin | DLGKKLLQAARAGQLDEVRELLKAGADVNAKDHYGWTPLHL |
| | #9 | repeat domain | AASEGHLEIVEVLLKAGADVNAKDWIGWTPLHLAASFGHLEIV |
| | | | EVLLKAGADVNAKDVSGKTPLHIAARQGHLEIVEVLLKAGADV |
| | | | NAQDKSGKTPADLAARAGHQDIAEVLQLQKAA |
| 10 | Binder | Designed ankyrin | DLGKKLLQAARAGQLDEVRELLKAGADVNAKDHYGWTPLHL |
| | #10 | repeat domain | AASEGHLEIVEVLLKAGADVNAKDWIGWTPLHLAASFGHLEIV |
| | | | EVLLKAGADVNAKDVSGKTPLHIAARQGHLEIVEVLLKAGADV |
| | | | NAQDKSGKTPADLAARAGHQDIAEVLQKAA |
| 11 | | CD3-specific | DLGQKLLEAAWAGQDDEVRILLAAGADVNAKNSRGWTPLHT |
| | | binding domain | AAQTGHLEIFEVLLKAGADVNAKNDKRVTPLHLAAALGHLEIV |
| | | | EVLLKAGADVNARDSWGTTPADLAAKYGHGDIAEVLQKAA |
| 12 | | CD3-specific | DLGQKLLEAAWAGQDDEVRELLKAGADVNAKDSQGWTPLH |
| | | binding domain | TAAQTGHLEIFEVLLKAGADVNAKDDKGVTPLHLAAALGHLEI |
| | | | VEVLLKAGADVNAQDSWGTTPADLAAKYGHEDIAEVLQKAA |
| 13 | | CD3-specific | DLGQKLLEAAWAGQDDEVRELLKAGADVNAKNSRGWTPLH |
| | | binding domain | TAAQTGHLEIFEVLLKAGADVNAKDDKGVTPLHLAAALGHLEI |
| | | | VEVLLKAGADVNAQDSWGTTPADLAAKYGHEDIAEVLQKAA |
| 14 | | CD3-specific | DLGQKLLEAAWAGQDDEVRELLKAGADVNAKNSRGWTPLH |
| | | binding domain | TAAQTGHLEIFEVLLKAGADVNAKNDKRVTPLHLAAALGHLEI |
| | | | VEVLLKAGADVNARDSWGTTPADLAAKYGHQDIAEVLQKAA |
| 15 | | CD3-specific | DLGQKLLEAAWAGQLDEVRILLKAGADVNAKNSRGWTPLHT |
| | | binding domain | AAQTGHLEIFEVLLKAGADVNAKTNKRVTPLHLAAALGHLEIV |
| | | | EVLLKAGADVNARDTWGTTPADLAAKYGHRDIAEVLQKAA |
| 16 | | Nucleic acid | GACTTAGGAAAGAAATTGCTGCAAGCCGCACGCGCCGGT |
| | | encoding a | CAACTTGATGAGGTGCGCGAATTATTGAAGGCAGGTGCAG |
| | | designed ankyrin | ACGTGAACGCTAAAGACTTTAAGGGACTTACTCCTTTACAC |
| | | repeat domain | TTAGCGGCCGCACATGGTCATTTGGAAATTGTGGAGGTCC |
| | | | TGTTGAAGGCTGGCGCCGACGTGAACGCCAAAGATGTTTA |
| | | | CGGTTGGACCCCATTACACATTGCTGCCGCCTCGGGACAT |
| | | | CTGGAAATTGTTGAGGTTCTGCTTAAAGCTGGCGCAGACG |
| | | | TTAATGCCAAGGACTGGTTGGGGATTACGCCCTTACACCT |
| | | | GGCCGCGTCACATGGACATTTAGAGATTGTAGAAGTCCTG |
| | | | TTAAAGGCGGGCGCGGACGTTAATGCCCAAGACAAAAGT |
| | | | GGCAAAACACCAGCGGATCTGGCCGCTCGTGCTGGACAC |
| | | | CAGGACATTGCTGAAGTGCTGCAGAAGGCAGCG |
| 17 | | Nucleic acid | GACTTAGGAAAGAAATTGCTGCAAGCCGCACGCGCCGGT |
| | | encoding a | CAACTTGATGAGGTGCGCGAATTATTGAAGGCAGGTGCAG |
| | | designed ankyrin | ACGTGAACGCTAAAGACTTTAAGGGACTTACTCCTTTACAC |
| | | repeat domain | TTAGCGGCCGAGCATGGTCATTTGGAAATTGTGGAGGTCC |
| | | | TGTTGAAGGCTGGCGCCGACGTGAACGCCAAAGATGTTTA |
| | | | CGGTTGGACCCCATTACACTGGGCTGCCGCCAAGGGACA |
| | | | TCTGGAAATTGTTGAGGTTCTGCTTAAAGCTGGCGCAGAC |
| | | | GTTAATGCCAAGGACTGGTTGGGGATTACGCCCTTACACC |
| | | | TGGCCGCGTCACATGGACATTTAGAGATTGTAGAAGTCCT |
| | | | GTTAAAGGCGGGCGCGGACGTTAATGCCCAAGACAAAAG |
| | | | TGGCAAAACACCAGCGGATCTGGCCGCTCGTGCTGGACA |
| | | | CCAGGACATTGCTGAAGTGCTGCAGAAGGCAGCG |
| 18 | | Nucleic acid | GACTTGGGGAAAAAACTGCTTCAGGCTGCACGCGCTGGT |
| | | encoding a | CAGTTAGATGAGGTGCGTGAGTTGTTAAAAGCTGGAGCGG |
| | | designed ankyrin | ACGTAAATGCTAAAGATCATTACGGATGGACACCCCTGCA |
| | | repeat domain | TCTTGCAGCGTCAGAAGGTCACCTTGAAATCGTCGAGGTC |
| | | | CTTTTGAAAGCAGGGGCAGATGTCAACGCCAAGGACTGGA |
| | | | TCGGTTGGACCCCTCTTCATTTAGCTGCTTCGTTCGGTCAT |
| | | | CTGGAGATTGTAGAAGTTTTATTAAAAGCCGGTGCGGATG |
| | | | TGAATGCAAAAGATGTCTCAGGGAAAACCCCGTTACACAT |
| | | | CGCCGCTCGTCAAGGGCATTTAGAGATCGTCGAGGTACTG |
| | | | TTGAAAGCGGGCGCAGATGTCAATGCACAGGACAAGTCC |
| | | | GGCAAAACTCCAGCGGATTTAGCTGCGCGCGCAGGACAC |
| | | | CAAGACATTGCGGAAGTCTTACAACTGCAGAAGGCAGCG |
| 19 | | Nucleic acid | GACTTGGGGAAAAAACTGCTTCAGGCTGCACGCGCTGGT |
| | | encoding a | CAGTTAGATGAGGTGCGTGAGTTGTTAAAAGCTGGAGCGG |
| | | designed ankyrin | ACGTAAATGCTAAAGATCATTACGGATGGACACCCCTGCA |
| | | repeat domain | TCTTGCAGCGTCAGAAGGTCACCTTGAAATCGTCGAGGTC |
| | | | CTTTTGAAAGCAGGGGCAGATGTCAACGCCAAGGACTGGA |
| | | | TCGGTTGGACCCCTCTTCATTTAGCTGCTTCGTTCGGTCAT |
| | | | CTGGAGATTGTAGAAGTTTTATTAAAAGCCGGTGCGGATG |
| | | | TGAATGCAAAAGATGTCTCAGGGAAAACCCCGTTACACAT |
| | | | CGCCGCTCGTCAAGGGCATTTAGAGATCGTCGAGGTACTG |
| | | | TTGAAAGCGGGCGCAGATGTCAATGCACAGGACAAGTCC |
| | | | GGCAAAACTCCAGCGGATTTAGCTGCGCGCGCAGGACAC |
| | | | CAAGACATTGCGGAAGTCCTGCAGAAGGCAGCG |
| 20 | | His-tag | MRGSHHHHHHGS |
| 21 | | N-terminal capping | DLGKKLLQAARAGQLDEVRELLKAGADVNA |
| | | module | |
| 22 | | N-terminal capping | DLGKKLLQAARAGQLDEVRILLKAGADVNA |
| | | module | |
| 23 | | N-terminal capping | DLGKKLLQAARAGQLDEVRILLAAGADVNA |
| | | module | |
| 24 | | N-terminal capping | DLGXKLLQAAXXGQLDEVRILLAAGADVNA |
| | | module | |
| | | (X represents any | |
| | | amino acid) | |
| 25 | | Ankyrin repeat | XDXXGXTPLHLAAXXGHLEIVEVLLKXGADVNA |
| | | module | |
| | | (X represents any | |
| | | amino acid) | |
| 26 | | Ankyrin repeat | KDXXGXTPLHLAAXXGHLEIVEVLLKAGADVNA |
| | | module | |
| | | (X represents any | |
| | | amino acid) | |
| 27 | | Ankyrin repeat | KDXXGXTPLHXAAXXGHLEIVEVLLKAGADVNA |
| | | module | |
| | | (X represents any | |
| | | amino acid) | |
| 28 | | C-terminal capping | QDKSGKTPADLAARAGHQDIAEVLQKAA |
| | | module | |
| 29 | | C-terminal capping | XDXXGXTPADXAARXGHQDIAEVLQKAA |
| | | module | |
| | | (X represents any | |
| | | amino acid) | |
| 30 | | Ankyrin repeat | KDFKGLTPLHLAAAHGHLEIVEVLLKAGADVNA |
| | | module | |
| 31 | | Ankyrin repeat | KDVYGWTPLHIAAASGHLEIVEVLLKAGADVNA |
| | | module | |
| 32 | | Ankyrin repeat | KDWLGITPLHLAASHGHLEIVEVLLKAGADVNA |
| | | module | |
| 33 | | Ankyrin repeat | KDVYGWTPLHWAAASGHLEIVEVLLKAGADVNA |
| | | module | |
| 34 | | Ankyrin repeat | KDVYGWTPLHWAAAKGHLEIVEVLLKAGADVNA |
| | | module | |
| 35 | | Ankyrin repeat | KDFKGLTPLHLAAEHGHLEIVEVLLKAGADVNA |
| | | module | |
| 36 | | Ankyrin repeat | KDVYGWTPLHWAAAKGHLEIVEVLLKAGADVNA |
| | | module | |
| 37 | | Ankyrin repeat | KDHYGWTPLHIAAQIGHLEIVEVLLKAGADVNA |
| | | module | |
| 38 | | Ankyrin repeat | KDWIGYTPLHLAASHGHLEIVEVLLKAGADVNA |
| | | module | |
| 39 | | Ankyrin repeat | KDVSGKTPLHVAAAHGHLEIVEVLLKAGADVNA |
| | | module | |
| 40 | | Ankyrin repeat | KDQYGWTPLHLAAYSGHLEIVEVLLKAGADVNA |
| | | module | |
| 41 | | Ankyrin repeat | KDWVGWTPLHLAASHGHLEIVEVLLKAGADVNA |
| | | module | |
| 42 | | Ankyrin repeat | KDEAGRTPLHIAAKQGHLEIVEVLLKAGADVNA |
| | | module | |
| 43 | | Ankyrin repeat | KDHYGWTPLHLAAAEGHLEIVEVLLKAGADVNA |
| | | module | |
| 44 | | Ankyrin repeat | KDWIGYTPLHIAASHGHLEIVEVLLKAGADVNA |
| | | module | |
| 45 | | Ankyrin repeat | KDSSGKTPLHIAAQHGHLEIVEVLLKAGADVNA |
| | | module | |
| 46 | | Ankyrin repeat | KDHYGWTPLHIAAQRGHLEIVEVLLKAGADVNA |
| | | module | |
| 47 | | Ankyrin repeat | KDWLGWTPLHVAASHGHLEIVEVLLKAGADVNA |
| | | module | |
| 48 | | Ankyrin repeat | KDLSGRTPLHIAARQGHLEIVEVLLKAGADVNA |
| | | module | |
| 49 | | Ankyrin repeat | KDHYGWTPLHLAASEGHLEIVEVLLKAGADVNA |
| | | module | |
| 50 | | Ankyrin repeat | KDWIGWTPLHLAASFGHLEIVEVLLKAGADVNA |
| | | module | |
| 51 | | Ankyrin repeat | KDVSGKTPLHIAARQGHLEIVEVLLKAGADVNA |
| | | module | |
| 52 | | Ankyrin repeat | xDxxGxTPLHLAxxxGxxxIVxVLLxxGADVNA |
| | | module | |
| | | (x represents any | |
| | | amino acid) | |
| 53 | | Ankyrin repeat | xDxxGxTPLHLAAxxGHLEIVEVLLKzGADVNA |
| | | module | |
| | | (x represents any | |
| | | amino acid) | |
| 54 | TCE #1 | TCE (without a | DLGKKLLEAARAGQDDEVRILMANGADVNALDWLGHTPLHL |
| | | half-life | AAYEGHLEIVEVLLKNGADVNAIDDNNGFTPLHLAAIDGHLEIV |
| | | extending moiety) | EVLLKNGADVNAQDKFGKTAFDISIDNGNEDLAEILQKAAGSP |
| | | | TPTPTTPTPTPTTPTPTPTGSDLGDKLLLAATSGQDDEVRILL |
| | | | AAGADVNAKDYDGDTPLHLAADEGHLEIVEVLLKAGADVNAK |
| | | | DYSGSTPLHAAAAYGHLEIVEVLLKAGADVNAQDVFGYTPAD |
| | | | LAAYVGHEDIAEVLQKAAGSPTPTPTTPTPTPTTPTPTPTGSD |
| | | | LGQKLLEAAWAGQDDEVRELLKAGADVNAKNSRGWTPLHT |
| | | | AAQTGHLEIFEVLLKAGADVNAKDDKGVTPLHLAAALGHLEIV |
| | | | EVLLKAGADVNAQDSWGTTPADLAAKYGHEDIAEVLQKAA |
| 55 | TCE #2 | TCE (with a half- | DLGKKLLEAARAGQDDEVRELLKAGADVNAKDYFSHTPLHLA |
| | | life extending | ARNGHLKIVEVLLKAGADVNAKDFAGKTPLHLAAADGHLEIVE |
| | | moiety) | VLLKAGADVNAQDIFGKTPADIAADAGHEDIAEVLQKAAGSPT |
| | | | PTPTTPTPTPTTPTPTPTGSDLGKKLLEAARAGQDDEVRILMA |
| | | | NGADVNALDWLGHTPLHLAAYEGHLEIVEVLLKNGADVNAID |
| | | | DNNGFTPLHLAAIDGHLEIVEVLLKNGADVNAQDKFGKTAFDI |
| | | | SIDNGNEDLAEILQKAAGSPTPTPTTPTPTPTTPTPTPTGSDL |
| | | | GDKLLLAATSGQDDEVRILLAAGADVNAKDYDGDTPLHLAAD |
| | | | EGHLEIVEVLLKAGADVNAKDYSGSTPLHAAAAYGHLEIVEVL |
| | | | LKAGADVNAQDVFGYTPADLAAYVGHEDIAEVLQKAAGSPTP |
| | | | TPTTPTPTPTTPTPTPTGSDLGQKLLEAAWAGQDDEVRELLK |
| | | | AGADVNAKNSRGWTPLHTAAQTGHLEIFEVLLKAGADVNAK |
| | | | NDKRVTPLHLAAALGHLEIVEVLLKAGADVNARDSWGTTPAD |
| | | | LAAKYGHQDIAEVLQKAA |

1. A composition comprising (i) a binding moiety and (ii) a drug molecule;
wherein said binding moiety reversibly binds to said drug molecule; and
wherein said binding moiety, when bound, inhibits a biological activity of said drug molecule.

2. The composition of claim 1, wherein said binding moiety comprises an antibody, an alternative scaffold, or a polypeptide.

3. The composition of claim 1, wherein said biological activity of said drug molecule is binding of said drug molecule to a biological target.

4. The composition of claim 1, wherein the binding affinity of said binding moiety to said drug molecule allows release of the drug molecule as a function of time upon administration of said composition to a mammal.

5. The composition of claim 1, wherein said binding moiety binds said drug molecule with a dissociation constant (K_{D}) of less than 10 nM.

6. The composition of claim 1, wherein said binding moiety has an off rate (k_{off}) from said drug molecule between about 1×10⁻⁸ s⁻¹ and about 1×10⁻⁴ s⁻¹.

7. The composition of claim 1, wherein said binding moiety has a blocking half-life (T_{1/2}) when complexed with said drug molecule, wherein said blocking half-life is calculated according to the following formula:
$$blocking {T}_{1 / 2} = \frac{ln ( 2 )}{{k}_{off}}$$

8. The composition of claim 7, wherein said blocking half-life (T_{1/2}) is at least about 2 hours.

9. The composition of claim 1, wherein said binding moiety comprises a designed ankyrin repeat domain.

10. The composition of claim 9, wherein said designed ankyrin repeat domain comprises an ankyrin repeat module comprising an amino acid sequence selected from the group consisting of (1) SEQ ID NOs: 30 to 51 and (2) sequences in which up to 9 amino acids in any of SEQ ID NOs: 30 to 51 are substituted by other amino acids.

11. The composition of claim 9, wherein said designed ankyrin repeat domain comprises an amino acid sequence selected from the group consisting of (1) SEQ ID NOs: 1 to 10 and (2) sequences that have at least 85% amino acid sequence identity with any of SEQ ID NOs: 1 to 10.

12. The composition of claim 1, wherein said drug molecule has binding specificity for CD3.

13. The composition of claim 1, wherein said drug molecule is a T-cell engager drug molecule (TCE).

14. The composition of claim 13, wherein binding of said binding moiety to said TCE drug molecule inhibits binding of said TCE drug molecule to T cells and/or activation of T cells.

15. A method for treating a disease, comprising administering to a subject in need thereof the composition of claim 1.

16. The composition of claim 1, wherein said drug molecule comprises a designed ankyrin repeat domain.

17. The composition of claim 16, wherein said designed ankyrin repeat domain has binding specificity for CD3.

18. The composition of claim 17, wherein said designed ankyrin repeat domain comprises an amino acid sequence selected from the group consisting of (1) SEQ ID NOs: 11 to 15 and (2) sequences that have at least 85% amino acid sequence identity with any of SEQ ID NOs: 11 to 15.

19. A composition comprising (i) a binding moiety and (ii) a drug molecule;
wherein said binding moiety reversibly binds to said drug molecule;
wherein said binding moiety, when bound, inhibits a biological activity of said drug molecule;
wherein the binding affinity of said binding moiety to said drug molecule allows release of the drug molecule as a function of time upon administration of said composition to a mammal;
wherein said binding moiety binds said drug molecule with a dissociation constant (K_{D}) of less than 10 nM;
wherein said binding moiety comprises a designed ankyrin repeat domain;
wherein said designed ankyrin repeat domain comprised in said binding moiety comprises an ankyrin repeat module comprising an amino acid sequence selected from the group consisting of (1) SEQ ID NOs: 30 to 51 and (2) sequences in which up to 9 amino acids in any of SEQ ID NOs: 30 to 51 are substituted by other amino acids;
wherein said drug molecule has binding specificity for CD3;
wherein said drug molecule comprises a designed ankyrin repeat domain; and
wherein said designed ankyrin repeat domain comprised in said drug molecule comprises an amino acid sequence selected from the group consisting of (1) SEQ ID NOs: 11 to 15 and (2) sequences that have at least 85% amino acid sequence identity with any of SEQ ID NOs: 11 to 15.

20. A method for treating a disease, comprising administering to a subject in need thereof the composition of claim 19.
